(19) United States
(12) Patent Application Publication
(10) Pub. No.: US 2005/0026235 A1
(43) Pub. Date: Feb. 3, 2005

(54) Fluorescent phospholipase assays and compositions

(76) Inventor: Ronald J. Graham, San Ramon, CA (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/877,421

(22) Filed: Jun. 25, 2004

Related U.S. Application Data
(60) Provisional application No. 60/484,041, filed on Jun. 30, 2003.

(30) Foreign Application Priority Data
Jun. 21, 2004 (WO) PCT/US04/20079

(51) Int. Cl. C12Q 1/42

(52) U.S. Cl. 435/21

(57) ABSTRACT

The present disclosure provides methods, compositions, and kits for carrying out phospholipase assays. In some aspects, the methods comprise the use of a lipid complex comprising a fluorescently-labeled phospholipase substrate and detecting an increase in fluorescence due to phospholipase-mediated cleavage of the substrate.

### 1. CROSS-REFERENCE TO RELATED CO-PENDING APPLICATIONS

This application is a continuation of International Application No. PCT/US04/______, entitled “FLUORESCENT PHOSPHOLIPASE ASSAYS AND COMPOSITIONS”, filed Jun. 21, 2004, having an attorney docket number of FP-72058-PC, and claims benefit of U.S. Provisional Application No. 60/484,041, filed Jun. 30, 2003, the disclosures of which are incorporated herein by reference.

### 2. FIELD

The present disclosure relates to methods and compositions using fluorogenic dyes for detecting or characterizing phospholipase enzymes.

### 3. INTRODUCTION

Phospholipids are the primary structural constituents of biological membranes. In addition to this structural role, the importance of phospholipids as mediators in cellular signaling processes has become increasingly apparent. Consequently, research into metabolic processes such as phospholipase action and lipid sorting and trafficking is rapidly expanding. The mechanism by which specificity of physiological responses are conferred by a limited number of signal transducing substances, typically enzymes, is poorly understood. Cellular receptors on the surfaces of various cells are involved and initiate multiple signaling pathways and are linked to G-protein. Many of these G-protein-linked receptors stimulate the activation of three phospholipases, phospholipase C (PLC), phospholipase D (PLD) and phospholipase A2 (PLA2).

Most phospholipids are derived from glycerol to which two fatty acyl residues (nonpolar or hydrophobic tails) and a single phosphorylalcohol substituent (polar head group) are attached. Head groups include phosphate (phosphatidic acid), as well as phosphate esters of choline, ethanolamine, glycerol and methanol. Sphingomyelins have the amino alcohol sphingosine instead of glycerol as the structural backbone.

Phospholipases are classified according to their site of action in the phospholipid molecule. Thus, a phospholipase A1 (PLA1) hydrolyzes the 1-acyl group of a phospholipid, i.e. it hydrolyzes the bond between the fatty acid and the glycerine residue at the 1-position of the phospholipid. A phospholipase A2 (PLA2) hydrolyzes the 2-acyl, or central acyl, group and phospholipases C (PLC) and D (PLD), which are also known as phosphodiesterases, cleave on different sides of the phosphodiester linkage.

The hydrolysis of a phospholipid by a PLA1 or a PLA2 results in the production of a so-called “lysophospholipid”. Selective hydrolysis of a phospholipid substrate with a PLA1 produces a 2-acyl lysophospholipid and selective hydrolysis of a phospholipid with a PLA2 results in the production of a 1-acyl lysophospholipid.

The phospholipase metabolites are involved in diverse cellular processes including signal transduction, host defense (including antibacterial effects), formation of platelet activating cofactor, membrane remodeling and general lipid metabolism (Dennis, J. Biol. Chem., 269:13057-13060 (1994); Dennis, Trends Biochem. Sci. 22:1-2 (1997)).

The primary substrates for PLC are the inositol containing lipids, specifically and typically phosphatidylinositol (PI). PLC belongs to a family of enzymes, also known as disulfide isomerases, which play a very important role in transmembrane signal transduction. Many extracellular signaling molecules including hormones, growth factors, neurotransmitters, and immunoglobulins bind to their respective cell surface receptors and activate PLCS. The role of an activated PLC is to catalyze the hydrolysis of phosphatidyl-inositol-4,5-bisphosphate (PIP2), a minor component of the plasma membrane to produce diacylglycerol and inositol 1,4,5-trisphosphate (IP3). Several distinct isoforms of PLC have been identified and are categorized as PLC-β, PLC-γ, and PLC-δ. PLCs have a molecular mass of 62-68 kDa, and their amino acid sequences show two regions of significant similarity. The first region (designated X) has about 170 amino acids, and the second (designated Y) contains about 260 amino acids.

The primary substrate for PLD and PLA2 is phosphatidylcholine (PC), a relatively ubiquitous constituent of cell membranes. PLD activity is present in plant and animal tissues. PLD activity has been detected in membranes and in cytosol. Biological characterization of PLD1 revealed that it could be activated by a variety of G-protein regulators, specifically PKC (protein kinase C), ADP-ribosylation factor (ARF), RhoA, Rac1 and cdc-42, either individually or together in a synergistic manner, suggesting that a single PLD participates in regulated secretion in coordination with ARF and in propagating signal transduction responses through interaction with PKC, PhoA and Rac1. PKC-independent PLD activation has been associated with Src and Ras oncogenic transformation (see Jiang, Mol. and Cell. Biol. 14:3676 (1994) and Morris, Trends in Pharmacological Sciences 17:182-85 (1996)).

The activity of cytosolic PLA2 on PC liberates arachidonic acid, a precursor for the biosynthesis of prostaglandins and leukotrienes and possible intracellular secondary messenger. PLA2s are a diverse class of enzymes with regard to function, localization, regulation, mechanism, structure and dependence on divalent metal ions for activity (Dennis (1994); Dennis (1997)). The PLA2s have been divided into ten groups (denoted using Roman numerals I through X) (Cupillard et al., J. Biol. Chem., 272:15745-15752 (1997); Dennis (1997)). The PLA2s of groups IV and VI are intracellular, high molecular weight enzymes which have not been as extensively studied as the secreted PLA2s (groups I-III, V and X) (Balsinde and Dennis, J. Biol. Chem. 272:16069-16072 (1997)). Group IV requires calcium for activity whereas the activity of group VI is calcium-independent.

The pathogenic fungus Cryptococcus neoformans secretes an enzyme termed “PLB”, containing phospholipase B (PLB), which removes both acyl chains from phospholipids.

A variety of assays for phospholipase activity have been described. In a widely used assay for PLC (catalog no. A-12218, Molecular Probes, Eugene, Oreg.) the activity is detected using 10-acetyl-3,7-dihydrophenoxazine (Amplex® Red reagent), a fluorogenic probe for H₂O₂. In a first step, PLC converts the phosphatidylcholine (lecithin) substrate to form phosphocholine and diacylglycerol. After the action of alkaline phosphatase, which hydrolyses phosphocholine, choline is oxidized by choline oxidase to betaine and H₂O₂. Finally, H₂O₂, in the presence of horseradish peroxidase, reacts with Amplex Red reagent in a 1:1 stoichiometry to generate the fluorescent product, resorufin. The assay is cumbersome, requiring the use of multiple enzymes and reagents which increases the cost and complexity of the system. The utility of the system for assessing modulators of phospholipase activity (i.e., inhibitors and activators) can be limited by the affect of such modulators on the accessory enzymes.

An assay for PLD activity in cell extracts was developed, utilizing a fluorogenic derivative of phosphatidylcholine as substrate. The reaction products were analyzed by thin-layer chromatography. (Ella et al., Anal. Biochem. 218:136-42 (1994)). An assay for serum PLA2 (Thuren, Clin. Chem. 31:714-7 (1985)) utilized liquid-liquid phase partition in the analysis of fluorogenic reaction products. These assays are laborious, requiring time-consuming post-reaction analyses.

There is a need for simple, direct and inexpensive assay systems for phospholipase activity. There is a need for methods and compositions which are suitable for high-throughput screening of phospholipases. There exists a need for assays for testing modulation of phospholipase activity by candidate compounds. 4. SUMMARY

In one aspect, the present disclosure provides a simple, homogeneous fluorogenic assay useful for detecting cleavage activity of a phospholipase. According to the method, a sample either known or suspected to contain a phospholipase is contacted with a lipid complex which comprises a labeled phospholipase cleavage substrate under conditions effective to permit the phospholipase to cleave the labeled phospholipase substrate. In some embodiments, the labeled phospholipase substrate comprises a hydrophobic moiety, a phosphate moiety and a fluorescent moiety. In some embodiments, the fluorescent moiety is covalently attached to the phosphate moiety, either directly or by way of an optional linker. In some embodiments, the fluorescent moiety is non-covalently attached to the phosphate moiety.

Following contact, a fluorescent signal in the sample is measured. An increase in the fluorescence signal is indicative of phospholipase cleavage activity. The fluorescence signal may be detected at one or more discrete time points following contact or, alternatively, the fluorescence signal may be detected substantially continuously as a function of time.

The assay may be used to detect the cleavage activity of any phospholipase, regardless of its point of cleavage. For example, the assay may be used to detect the cleavage activity of a PLA1, a PLA2, a PLB, a PLC or a PLD.

The assay may be used in a variety of different contexts. In some embodiments, the assay may be used to detect the presence or absence of a phospholipase in a sample of interest, such as a column chromatography fraction, a cell or tissue lysate or other biological sample such as a blood sample. Such assays can be used, for example, to determine whether a specific cell or tissue type of interest expresses a phospholipase.

In some embodiments, the assay may be used to screen a plurality of phospholipases, such as a plurality of mutant phospholipases, for cleavage activity. Such assays are useful, for example, for determining the amino acid residues responsible for the cleavage activity of a particular phospholipase, or for identifying phospholipases having altered cleavage activity as compared to known phospholipases.

In another aspect, the disclsoure provides methods of identifying compounds that modulate the cleavage activity of a phospholipase. According to the method, a phospholipase is contacted with a lipid complex comprising a labeled phospholipid cleavage substrate, as described above, in the presence of a candidate compound. Following contact, the fluorescence signal of the fluorescent moiety of the labeled phospholipase substrate is detected, either at one or more discrete time points or continuously, as described above. Comparison of the fluorescence signal with that of a control assay carried out in the absence of the candidate compound reveals whether the candidate compound modulates the cleavage activity of the phospholipase. An increase in the fluorescence signal as compared to the control identifies the compound as an activator of the phospholipase, whereas a decrease in the fluorescence signal identifies the compound as an antagonist (inhibitor) of the phospholipase. The candidate compound can be added at various concentrations in order to obtain kinetic parameters for the compound. In some embodiments, a candidate compound is contacted with a phospholipase prior to exposure of the phospholipase with the labeled phospholipase cleavage substrate.

The assay can be used to screen any type of candidate compound, ranging from small organic compounds to large biological compounds such as proteins, for phospholipase modulating activity. The methods are amenable to automation, and may find particular use in automated high throughput screens designed to identify phospholipase modulating compounds useful as drug leads or as therapeutic agents.

In another aspect, the disclosure provides compositions and kits useful for carrying out the methods of the disclosure. In some embodiments, a lipid complex composition is provided which comprises a labeled phospholipase cleavage substrate as previously described. The labeled phospholipase substrate is included in the lipid complex in an amount sufficient to result in quenching of the fluorescence signal of the fluorescent moiety of the phospholipase substrate as compared to its signal when released from the complex via cleavage by a phospholipase. The composition may be provided alone, or as one component of a kit useful for carrying out the methods of the disclosure. When included as a part of a kit, the kit may optionally include additional components, such as one or more phospholipases, sample buffers, inhibitors, etc.

### 5. BRIEF DESCRIPTION OF THE DRAWINGS

Aspects of the disclosure can be more fully understood with respect to the following drawings:

FIG. 1 illustrates an embodiment of a fluorescently-labeled phospholipase substrate;

FIG. 2 schematically illustrates one embodiment of a lipid complex of the disclosure;

FIG. 3 schematically illustrates another embodiment of a lipid complex of the disclosure;

FIG. 4 illustrates phospholipase-mediated release of a fluorescent dye from a fluorescently-labeled phospholipase substrate integrated into a lipid complex;

FIG. 5 schematically illustrates the synthesis of a fluorescently labeled phospholipase substrate;

FIG. 6 schematically illustrates PLC-mediated cleavage of a fluorescently-labeled phospholipase substrate;

FIG. 7 shows data from a continuous fluorogenic assay of PLC;

FIG. 8 shows a plot of initial rate vs. concentration for the PLC-mediated cleavage of a fluorescently-labeled phospholipase substrate;

FIG. 9 illustrates the synthesis of a fluorescently labeled phospholipase substrate;

FIG. 10 schematically illustrates PLC-mediated cleavage of a fluorescently-labeled phospholipase substrate;

FIG. 11 illustrates a structure of an inhibitor of PLC;

FIG. 12 shows the effect of an inhibitor on the PLC-mediated cleavage of a fluorescently-labeled phospholipase substrate;

FIG. 13 shows a dose-response curve for inhibition of PLC activity by the inhibitor shown in FIG. 11;

FIG. 14 shows a Dixon plot of the data from FIG. 13;

FIG. 15 schematically illustrates PLA2-mediated cleavage of a fluorescently-labeled phospholipase substrate;

FIG. 16 shows a continuous fluorogenic PLA2 assay using a fluorescently-labeled phospholipase substrate;

FIG. 17 shows a plot of initial rate vs. concentration for the PLA2-mediated cleavage of a fluorescently-labeled phospholipase substrate;

FIG. 18 schematically illustrates PLA2-mediated cleavage of a fluorescently-labeled phospholipase substrate;

FIG. 19 illustrates structures of a fluorescently-labeled phospholipase substrate and an exemplary inhibitor of PLA2 activity;

FIG. 20 shows a dose-response curve for inhibition of PLA2 activity by a PLA2 inhibitor;

FIG. 21 shows initial and final fluorescence spectra for a fluorescently-labeled phospholipase substrate integrated into a lipid complex and subjected to phospholipase activity;

FIG. 22 shows data from a continuous fluorogenic assay using a fluorescently-labeled phospholipase substrate integrated into a lipid complex; and

FIG. 23 shows a plot of initial rate vs. substrate concentration for a fluorescently-labeled phospholipase substrate integrated into a lipid complex.

### 6. DETAILED DESCRIPTION

It is to be understood that both the foregoing general description and the following detailed description are exemplary and explanatory only and are not restrictive of the disclosure. In this application, the use of the singular includes the plural unless specifically state otherwise. Also, the use of “or” means “and/or” unless stated otherwise. Similarly, “comprise”, “comprises”, “comprising”, “include”, “includes” and “including” are not intended to be limiting.

6.1 Definitions

As used herein, the following terms are intended to have the following meanings:

“Quench” refers to a reduction in the fluorescence intensity of a fluorescent group as measured at a specified wavelength, regardless of the mechanism by which the reduction is achieved. As specific examples, the quenching may be due to molecular collision, energy transfer such as FRET, a change in the fluorescence spectrum (color) of the fluorescent group or any other mechanism. The amount of the reduction is not critical and may vary over a broad range. The only requirement is that the reduction be measurable by the detection system being used. Thus, a fluorescence signal is “quenched” if its intensity at a specified wavelength is reduced by any measurable amount.

“Lipid complex” refers to any complex formed when a lipid is placed in aqueous solution. Such complexes can include micelles, vesicles (including multilamellar and unilamellar) or any other type of complexes as are know in the art. Specifically included in the definition of lipid complexes are liposomes, which are self-closed vesicles where one or several lipid membranes encapsulate part of the solvent.

“Homogeneous assay” refers to a method carried out entirely in the liquid phase. Once the components of the assay have been combined, there is no need to remove contents of the assay to determine the result.

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In some embodiments, the alkyl groups are (C1-C6) alkyl.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In some embodiments, the aryl group is (C5-C15) aryl, with (C5-C10) being even more preferred. Particularly preferred aryls are cyclopentadienyl, phenyl and naphthyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In some embodiments, the arylalkyl group is (C6-C21) arylalkyl, e.g., the alkanyl, alkenyl or alkynyl moiety of the arylalkyl group is (C1-C6) and the aryl moiety is (C5-C15). In some embodiments the arylalkyl group is (C6-C13), e.g., the alkanyl, alkenyl or alkynyl moiety of the arylalkyl group is (C1-C3) and the aryl moiety is (C5-C10).

6.2 Exemplary Embodiments

The present disclosure provides compositions, methods and kits that utilize certain fluorescently-labeled phospholipid cleavage substrates. The labeled phospholipid cleavage substrates are generally phospholipids in which the polar head group includes a fluorescent moiety. The fluorescently-labeled phospholipid cleavage substrate therefore generally comprises a hydrophobic moiety, a phosphate moiety and a fluorescent moiety. The fluorescent moiety is covalently attached to the phosphate moiety, either directly or by way of an optional linker.

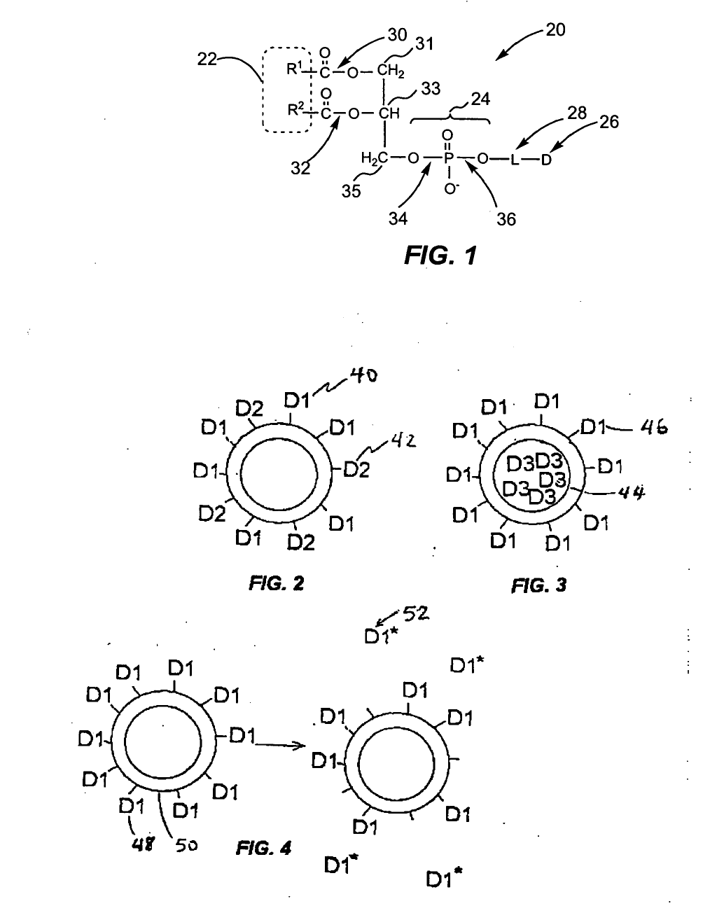

FIG. 1 illustrates a generalized embodiment of a non-limiting example of a labeled phospholipase cleavage substrate 20. The labeled phospholipase cleavage substrate generally comprises a hydrophobic moiety 22, a phosphate moiety 24 and a fluorescent moiety or fluorophore “D” 26. The fluorescent moiety is attached to the phosphate moiety, either directly or by way of an optional linker “L” 28. Hydrolytic cleavage sites for PLA1, PLA2, PLC and PLD are shown at 30, 32, 34 and 36, respectively.

The hydrophobic moiety 22 can be any combination of atoms that will function to integrate the labeled phospholipase substrate into a lipid complex but that will not interfere with a phospholipase assay as described herein. The hydrophobic moiety can include one or more hydrophobic tails, such as R1 and R2, as shown in FIG. 1, wherein R1 and R2 can be the same or different. The exact length, size and/or composition of each hydrophobic tail can be selectively varied. In some embodiments, the hydrophobic moiety comprises at least one hydrocarbon of sufficient hydrophobic character (e.g., length and/or size) to cause the labeled phospholipase substrate including it to become integrated or incorporated into a lipid complex when the labeled phospholipase substrate, either alone or in combination with other phospholipid compounds such as described hereinbelow, is placed in an aqueous environment at a concentration above a complex-forming threshold, such as above the critical micelle concentration (CMC). Thus, each hydrophobic tail may comprise alkyl, aryl, or arylalkyl groups, or combinations of such groups. The alkyl groups, or the alkyl portion of the arylalkyl groups, may be saturated or unsaturated, and may be linear, branched, cyclic or combinations of linear, branched and cyclic, in conformation. The aryl groups or the aryl portions of the arylalkyl groups may be monocyclic, polycyclic or bi-, or tri-, cyclic, etc. The hydrophobic moiety may comprise two, three or even more hydrophobic tails. The carbon atoms comprising the hydrophobic moiety may be substituted with various substituents.

The chains comprising the hydrophobic moiety may be attached to the remainder of the molecule via virtually any type of linkage, provided that the resultant labeled phospholipase cleavage substrate is cleavable by the particular phospholipase being assessed. As illustrated in FIG. 1, phospholipases A1 and A2 cleave phospholipids at the ester linkages 30 and 32, respectively, connecting the chains R¹ and R² of the hydrophobic moiety 22 to the remainder of the molecule. Thus, in embodiments in which phospholipase A1 and A2 are being assayed or assessed, carboxylic ester linkages such as those illustrated in FIG. 1 may be preferred. However, embodiments useful for identifying enzymes capable of cleaving phospholipids having alternative linkages at one or both of these positions, such as thioester, amide, sulfonamide, carbamate or other linkages may also be employed. Unlike phospholipases A1 and A2, phospholipases C and D cleave phospholipids at phosphate bonds 34 and 36, respectively.

In an exemplary embodiment, illustrated in FIG. 1, the labeled phospholipase substrate includes a hydrophobic moiety composed of two hydrophobic tails, R¹ and R², attached to the C1 (31) and C2 (33) of a glycerol “backbone” via ester linkages. In this illustrative embodiment, R¹ and R² may be any of the previously-described substituted or unsubstituted hydrocarbon groups. In some embodiments, R1 and R2 are each the same or different saturated or unsaturated C6,C7,C8,C9,C10,C11, C12, C13,C14,C15, C16, C17, C18, C19, C20, C22, C24 or C26 n-alkyl chain.

In one example, the R¹and R² chains are selected from amongst the alkyl chains commonly known to comprise phospholipids such that the hydrophobic moiety of the labeled phospholipase substrate corresponds to the hydrophobic moiety of a naturally occurring phospholipid. Non-limiting examples of suitable n-alkyl chains include those derived from commonly occurring fatty acids, such as the fatty acids provided in Table 1, below.

TABLE 1

| Length:no. of unsaturated | |
|---|---|
| C—C bonds | Common name |
| 14:0 | myristic acid |
| 16:0 | palmitic acid |
| 18:0 | stearic acid |
| 18:1 cisΔ⁹ | oleic acid |
| 18:2 cisΔ^{9,12} | linoleic acid |
| 18:3 cisΔ^{9,12,15} | linonenic acid |
| 20:4 cisΔ^{5,8,11,14} | arachidonic acid |
| 20:5 cisΔ^{5,8,11,14,17} | eicosapentaenoic acid |
| | (an omega-3 fatty acid) |

Referring again to FIG. 1, the fluorescent moiety 26 may be any entity that provides a fluorescent signal that can be used to follow phospholipase-mediated cleavage as described herein. The fluorescent moiety may be any fluorescent dye or fluorophore having the following properties: it is capable of producing a detectable fluorescence signal in the assay medium; the fluorescence signal can be “self-quenched” when the labeled phospholipase substrate is included in a lipid complex; and the fluorescent dye is capable of fluorescing in an aqueous medium. Fluorescent dyes having these properties include by way of example and not limitation, xanthenes such as fluoresceins, rhodamines and rhodols, cyanines, phthalocyanines, squairanines, and bodipy dyes. An example of an unsuitable dye is a dansyl.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings: 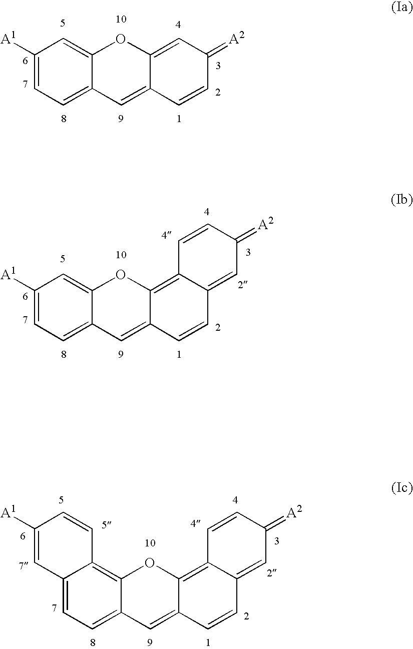
In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In some embodiments, substituents include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or —Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆ ) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀) aryleno bridges. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below: 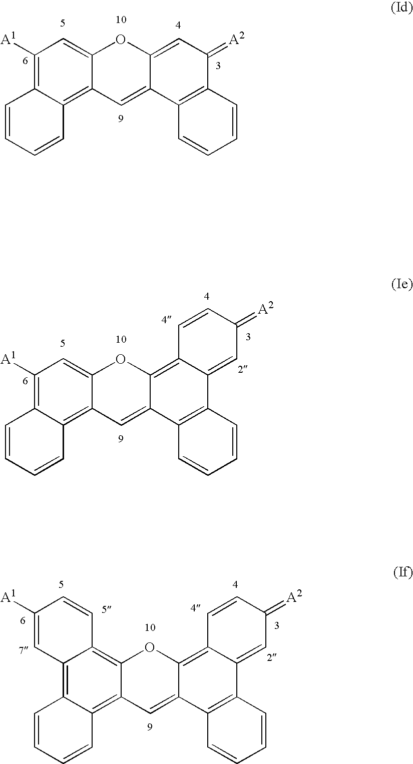
The benzo or aryleno bridges may be substituted at one or more positions with a variety of different substituent groups, such as the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that comprise bridges involving the exocyclic nitrogens are illustrated below: 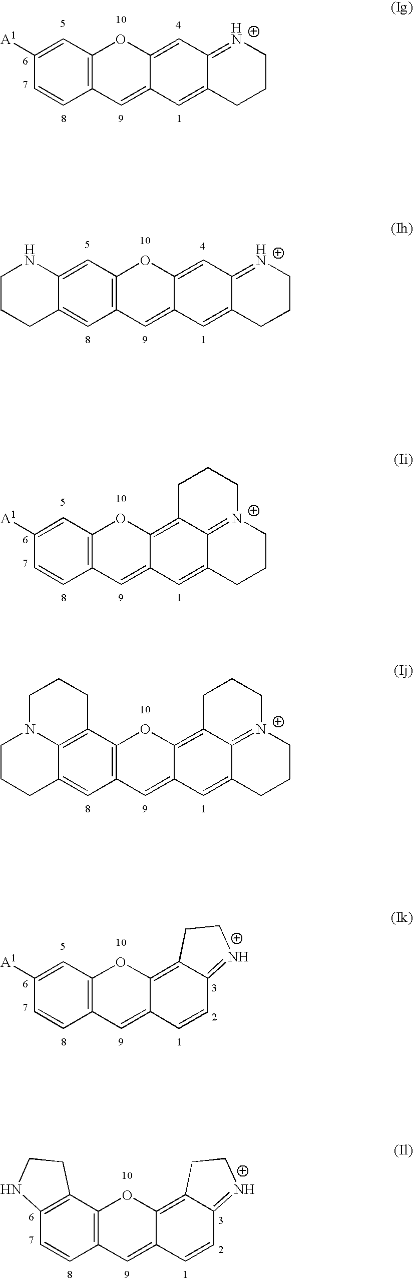
The parent ring may also comprise a substituent at the C9 position. In some embodiments, the C9 substituent is selected from acetylene, lower (e.g., from 1 to 6 carbon atoms) alkanyl, lower alkenyl, cyano, aryl, phenyl, heteroaryl, electron-rich heteroaryl and substituted forms of any of the preceding groups. In embodiments in which the parent ring comprises benzo or aryleno bridges fused to the C1/C2 and C7/C8 positions, such as, for example, rings (Id), (Ie) and (If) illustrated above, the C9 carbon is preferably unsubstituted.

In some embodiments, the C9 substituent is a substituted or unsubstituted phenyl ring such that the xanthene dye comprises one of the following structures: 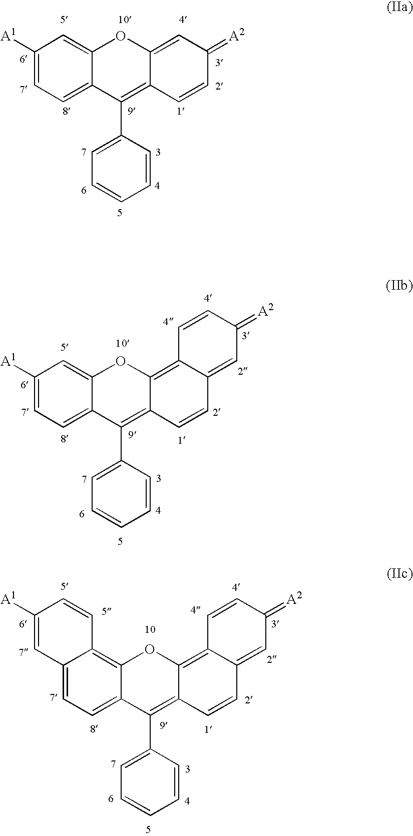
The carbons at positions 3, 4, 5, 6 and 7 may be substituted with a variety of different substituent groups, such as the substituent groups previously described for carbons C1-C8. In some embodiments, the carbon at position C3 is substituted with a carboxyl (—COOH) or sulfuric acid (—SO₃H) group, or an anion thereof. Dyes of formulae (IIa), (IIb) and (IIc) in which A¹ is OH and A² is O are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (IIb) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent moiety 26 comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichlorotetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In some embodiments, the fluorescent moiety comprises a 4,7-dichloro-orthocarboxyrhodamine dye.

In some embodiments, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In some embodiments, the fluorescein moiety comprises a 4,7-dichloro-orthocarboxyfluorescein dye.

In some embodiments, the dye can be a cyanine, phthalocyanine, squaraine, or bodipy dye, such as described in the following references and references cited therein: U.S. Pat. Nos. 5,863,727, 5,800,996, 5,945,526, 6,080,868, 5,436,134, 5,863,753, 6,005,113 and PCT Publication WO 96/04405.

In some embodiments, the dye comprises a dye pair or higher order network, such as described in U.S. Pat. Nos. 5,800,996, 5,863,727, 6,130,073, 6,232,075 and 6,399,392. In general, the dye pair can include a donor dye which absorbs light at a first wavelength and emits excitation energy in response, and acceptor dye which is capable of absorbing the excitation energy emitted by the donor dye and fluorescing at a second wavelength in response, and a linker which attaches the donor dye to the acceptor dye. The members of the dye pair are positioned such that they can undergo energy transfer. A specific example of such a pier if a FRET dye pair.

A fluorescent dye can be synthesized (or purchased) which includes a reactive group at one of the substituent positions for coupling of the dye to a phospholipid. Such a dye can be reacted with a lipid that includes a functional group with suitable reactivity, i.e., a complementary functionality. The point of attachment of a reactive group to the fluorescent dye can be any position that does not interfere with the ability of the labeled phospholipase substrate that is formed to function as a substrate as described herein. In non-limiting examples, for xanthene dyes, a reactive group can be attached to C9 phenyl (if present), a rhodamine exocyclic ring nitrogen, or via a 4-aminomethyl group.

In the instant substrate compounds, the fluorescent moiety and the lipid can be attached in any way that permits them to perform their respective functions, and can be attached via an optional linker. The chemical composition of a linker between the fluorescent moiety and the lipid of the substrate compound is not critical for success. Choosing a linker having properties suitable for a particular application is within the capabilities of those having skill in the art. The linker can comprise any combination of atoms that will function to connect the fluorescent dye with the lipid but will not interfere with an enzyme assay as described herein. In some embodiments, the fluorescent moiety can be covalently linked to the to the polar head group of a phospholipid either directly or by way of an optional linker.

In FIG. 1, “L” at 28 represents a linker that is either a chemical linkage of one or more atoms, or a bond without linkage atoms. The linker can comprise any combination of atoms that will function as a linker but will not interfere with a phospholipase assay as described herein. In some embodiments, the linker can incorporate a portion of a phospholipid. For example, when a labeled phospholipase substrate is prepared from a phospholipid having a polar head group, the particular head group, and the design of a reactive group in the fluorescent dye will dictate what “L” will be. The linker can be formed from the reaction of a reactive group in the polar head group (or a polar head group that has been modified to include a suitable reactive group) with a “complementary” reactive group attached to a fluorescent dye as indicated above. In one example, the linker is derived from the reaction of the primary amine in a phosphatidylethanolamine head group with a succinimidyl ester that is attached to a fluorescent dye as described in the Examples hereinbelow. In other examples, the polar head group can include other reactive groups, such as serine, hydroxyl, and thiol.

A general example of the preparation of a fluorescently-labeled phospholipase substrate showing the coupling of a phospholipid having a reactive functional group, R^{x}, with an activated fluorescent dye, having a complementary functional group, F^{x}, is shown in the following Scheme (I): 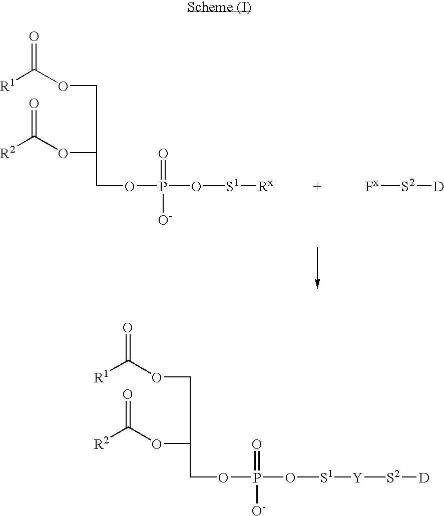
Covalent conjugates are obtained by reacting a fluorescent dye including a reactive group F^{x} with a lipid that contains, or is modified to contain, a functional group R^{x} that is complementary to reactive group F^{x}.

The exact identities of R^{x} and F^{x} will depend upon the nature of the desired covalent linker and the chemistry used to form the covalent linker. Generally, reactive group R^{x} is a functional group that is capable of reacting with a complementary functional group F^{x} under specified reaction conditions to form a covalent linker. However, those of skill in the art will recognize that a variety of functional groups that are typically unreactive under certain reaction conditions can be activated to become reactive. Groups that can be activated to become reactive include, e.g., carboxylic acids and esters, including salts thereof. Such groups are referred to herein as “activatable precursors” and are specifically intended to be included within the expression “reactive group.”

Pairs of reactive groups R^{x} and complementary groups F^{x} suitable for forming covalent linker with one another under a variety of different reaction conditions are well-know. Any of these complementary pairs of groups can be used to covalently conjugate the dyes of the disclosure to lipids as described herein. In some embodiments, reactive group R^{x} and complementary functional group F^{x} comprise complementary electrophiles and nucleophiles (or their respective activatable precursors). In some embodiments, reactive group R^{x} is a photoactivatable group that becomes chemically reactive only after illumination with light of an appropriate wavelength and complementary functional group F^{x} is a group capable of forming a covalent linker with the chemically reactive species.

A plethora of complementary electrophile/nucleophile pairs and photoactivatable groups suitable for covalently conjugating two molecules together are well-known. The actual choice of complementary pairs and/or photoactivatable group will depend upon a variety of factors, and will be apparent to those of skill in the art. Examples of complementary electrophiles and nucleophiles suitable for use in a wide variety of contexts are shown in the following Table 2, where reaction between the indicated electrophilic and nucleophilic species yields the indicated covalent linkage. Conditions under which the covalent linkages may be formed are well-known.

TABLE 2

| Electrophilic Group | Nucleophilic Group | Resultant Covalent Linkage |
|---|---|---|
| activated esters* | Amines/anilines | carboxamides |
| Acyl azides** | Amines/anilines | carboxamides |
| Acyl halides | Amines/anilines | carboxamides |
| Acyl halides | alcohols/phenols | esters |
| Acyl nitriles | alcohols/phenols | esters |
| Acyl nitriles | Amines/anilines | carboxamides |
| aldehydes | Amines/anilines | imines |
| aldehydes or ketones | Hydrazines | hydrazones |
| aldehydes or ketones | Hydroxylamines | oximes |
| alkyl halides | Amines/anilines | alkyl amines |
| alkyl halides | carboxylic acids | esters |
| alkyl halides | Thiols | thioethers |
| alkyl halides | alcohols/phenols | ethers |
| alkyl sulfonates | Thiols | thioethers |
| alkyl sulfonates | carboxylic acids | esters |
| alkyl sulfonates | alcohols/phenols | esters |
| anhydrides | alcohols/phenols | esters |
| anhydrides | Amines/anilines | caroboxamides |
| Aryl halides | Thiols | thiophenols |
| Aryl halides | Amines | aryl amines |
| aziridines | Thiols | thioethers |
| boronates | Glycols | boronate esters |
| carboxylic acids | Amines/anilines | carboxamides |
| carboxylic acids | Alcohols | esters |
| carboxylic acids | Hydrazines | hydrazides |
| carbodiimides | carboxylic acids | N-acylureas or anhydrides |
| diazoalkanes | carboxylic acids | esters |
| epoxides | Thiols | thioethers |
| haloacetamides | Thiols | thioethers |
| halotriazines | Amines/anilines | aminotriazines |
| halotriazines | alcohols/phenols | triazinyl ethers |
| imido esters | Amines/anilines | amidines |
| isocyanates | Amines/anilines | ureas |
| isocyanates | alcohols/phenols | urethanes |
| isothiocyanates | Amines/anilines | thioureas |
| maleimides | Thiols | thioethers |
| phosphoramidites | Alcohols | phosphate esters |
| Silyl halides | Alcohols | silyl ethers |
| sulfonate esters | Amines/anilines | alkyl amines |
| sulfonate esters | Thiols | thioethers |
| sulfonate esters | carboxylic acids | esters |
| sulfonate esters | Alcohols | esters |
| sulfonyl halides | Amines/anilines | sulfonamides |
| sulfonyl halides | phenols/alcohols | sulfonate esters |

*Activated esters, as understood in the art, generally have the formula —C(O)Z, where Z is, a good leaving group (e.g., oxysuccinimidyl, oxysulfosuccinimidyl, 1-oxybenzotriazolyl, etc.).
**Acyl azides can rearrange to isocyanates.

Additional examples of complementary functional groups are described in U.S. Pat. Nos. 5,231,191, 5,227,487, 6,248,884and 6,716,994.

Exemplary photoactivatable groups suitable for conjugation via light-activated cross-linking include, but are not limited to, azido, 4-azido-phenyl and 2-nitro-4-azido-phenyl. Conjugation using photoactivatable groups typically-involves illuminating a mixture comprising the photoactivatable dyes and the molecule or substance to be conjugated, followed by separation of unreacted dyes and byproducts.

In some embodiments, reactive group F^{x} is a group that reacts with, or that can be readily activated to react with, an amine, a thiol or an alcohol. A preferred reactive group F^{x} capable of reacting with a hydroxyl is a phosphoramidite. A preferred reactive group F^{x} capable of reacting with an amine is a carboxylic acid or an activated ester, most preferably a N-hydroxysuccinimidyl (NHS) ester. The NHS ester may be conveniently obtained by reacting a dye including a carboxylic acid reactive group F^{x} with N-hydroxysuccinimide in the presence of an activating agent (e.g., dicyclohexylcarbodiimide ) according to known methods.

For a discussion of the various reactive groups and respective complementary functional groups that can be conveniently used to covalently conjugate fluorescent dyes to lipids, as well as reaction conditions under which the conjugation reactions can be carried out, see Haugland, 1996, Molecular Probes Handbook of Fluorescent Probes and Research Chemicals, Molecular Probes, Inc.; Brinkley, 1992, Bioconjugate Chem. 3:2 and Garman, 1997, Non-Radioactive Labelling: A Practical Approach, Academic Press, London, as well as the references cited in all of the above. Additional suitable groups can be found in U.S. Pat. No. 5,268,486 (see, e.g., Col. 15-17).

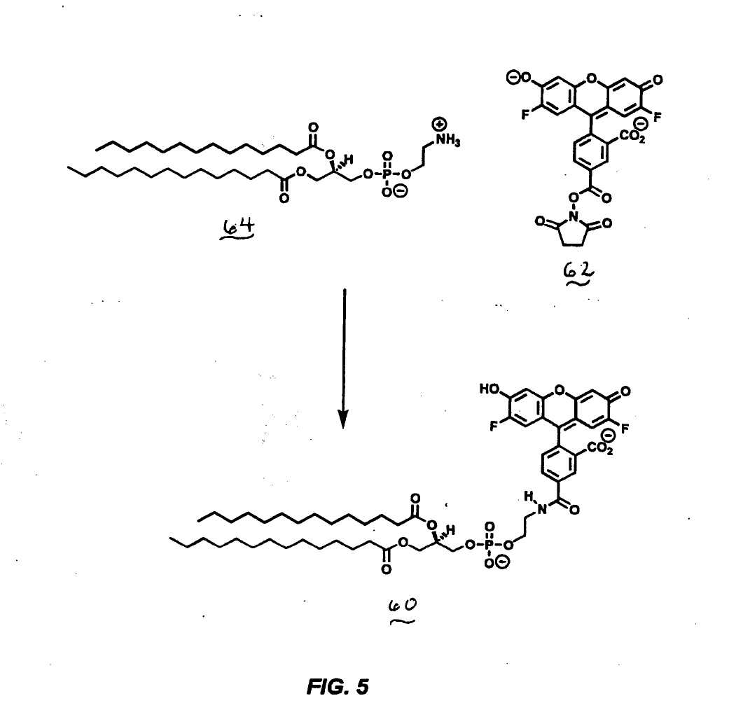

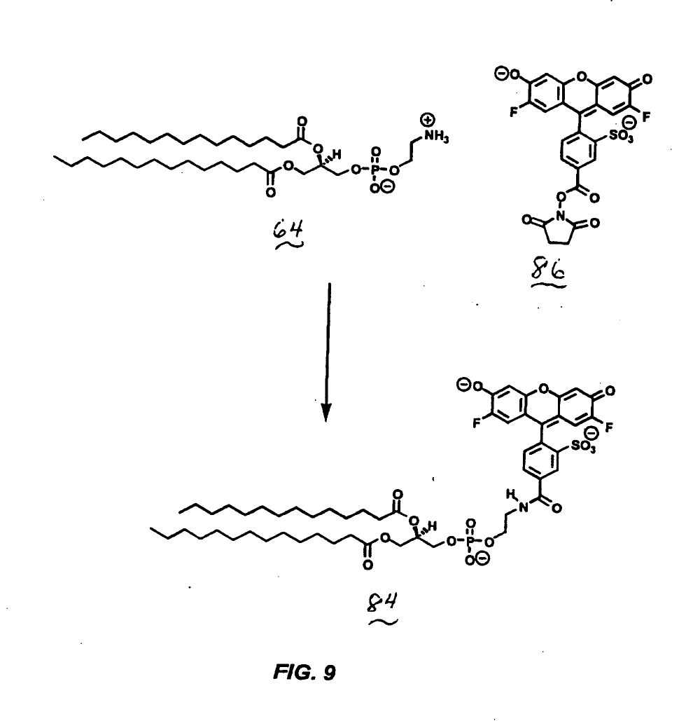

Particular examples of a reaction to form a fluorescently labeled phospholipase substrate are shown in FIGS. 5 and 9 as described hereinbelow.

Referring to Scheme I and FIG. 1, an exemplary embodiment of a linker “L” in a phospholipase substrate is illustrated by the structure shown in the following formula:
—S¹—Y—S²—
where S¹ and S² are spacing moieties. S¹ and S² can be the same length or different lengths, and typically comprise atoms such as C, H, N, S and O or groups that are conventionally used to space one group from another.

The length and chemical composition of these spacing moieties can be selectively varied. In some embodiments, the spacing moieties can be selected to have specified properties. For example, a spacing moiety can be hydrophobic in character, hydrophilic in character, long or short, rigid, semirigid or flexible, depending upon the particular application. The spacing moiety can be optionally substituted with one or more substituents or one or more groups for the attachment of additional substituents, which may be the same or different, thereby providing a “polyvalent” capable of conjugating additional molecules or substances. In some embodiments, however, the a moiety does not comprise such additional substituents.

Choosing a spacing moiety having properties suitable for a particular application is within the capabilities of those having skill in the art. A spacing moiety can be hydrophilic or hydrophobic, long or short, rigid, semirigid or flexible. A spacing moiety may comprise an alkyl, aryl, or arylalkyl moiety. The alkyl moiety, or the alkyl portion of the arylalkyl moiety, may be saturated or unsaturated, and may be linear, branched, cyclic or combinations of linear, branched and cyclic, in conformation. The aryl moiety or the aryl portions of the arylalkyl moiety may be monocyclic, polycyclic or bi-, or tri-, cyclic, etc. Carbon atoms comprising the spacing moiety may be substituted with various substituents. In some embodiments, a spacing moiety can be a straight chain hydrocarbon or a cyclic hydrocarbon. In some embodiments, a spacing moiety can comprise an ether, an ester, a thioether, a thioester,. a phosphoester or combination thereof. In some embodiments, a spacing moiety can be —(CH₂)ₓ—, where x can range from 1 to 10. For example, where a rigid spacing moiety is desired, it may comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Where a flexible spacing moiety is desired, it may comprise a flexible polypeptide such as polyglycine or a flexible saturated alkanyldiyl or heteroalkanyldiyl. Hydrophilic spacing moieties may comprise, for example, polyalcohols or polyethers such as polyalkyleneglycols, or other spacers. Hydrophobic spacing moieties may comprise, for example, alkyldiyls or aryldiyls. S¹ can include a portion of a polar head group. S² can include a portion of a fluorescent dye or a reactive group attached thereto. “Y” is a linkage moiety, and can comprise, for example, an ester, an ether, a thioester, a thioether or an amide. In some embodiments, when the phospholipid head group comprises ethanolamine, the spacing moiety S¹ comprises —(CH₂)₂— and R^{x} comprises —NH₂. Specific examples of —S¹—Y—S²— include:
—(CH₂)₂NH—, in which Y is —NH— and S¹ is —(CH₂)₂—;
—(CH₂)₂NH—SO₂—, in which S¹ is —(CH₂)₂—, Y is —NH—, and S² is —SO₂—; and
—CH₂CH(CO₂H)NH—SO₂—, in which S¹ is —(CH₂)CH—, Y is —(CO₂H)NH—, S² is —SO₂—.

The substrate compounds of the disclosure can be readily formed by synthetic methods known in the art. Fluorescent dyes that can be used to prepare the substrate compound can be prepared synthetically using conventional methods or purchased commercially (e.g. Sigma-Aldrich and/or Molecular Probes). Non-limiting examples of suitably reactive fluorescent dyes that are commercially available from Molecular Probes (Eugene, Oreg.) are provided in Table 3, below:

TABLE 3

| Catalog | |
|---|---|
| Number | Product Name |
| C-20050 | 5-carboxyfluorescein-bis-(5-carboxymethoxy-2-nitrobenzyl) |
| | ether, -alanine-carboxamide, succinimidyl ester (CMNB- |
| | caged carboxyfluorescein, SE) |
| C-2210 | 5-carboxyfluorescein, succinimidyl ester (5-FAM, SE) |
| C-1311 | 5-(and-6)-carboxyfluorescein, succinimidyl ester (5(6)-FAM, |
| | SE) |
| D-16 | 5-(4,6-dichlorotriazinyl) aminofluorescein (5-DTAF) |
| F-6106 | 6-(fluorescein-5-carboxamido)hexanoic acid, succinimidyl |
| | ester (5-SFX) |
| F-2182 | 6-(fluorescein-5-(and-6)-carboxamido) hexanoic acid, |
| | succinimidyl ester (5(6)-SFX) |
| F-6129 | 6-(fluorescein-5-(and-6)-carboxamido) hexanoic acid, |
| | succinimidyl ester (5(6)-SFX) |
| F-6130 | fluorescein-5-EX, succinimidyl ester |
| F-143 | fluorescein-5-isothiocyanate (FITC ‘Isomer I’) |
| F-1906 | fluorescein-5-isothiocyanate (FITC ‘Isomer I’) |
| F-1907 | fluorescein-5-isothiocyanate (FITC ‘Isomer I’) |
| F-144 | fluorescein-6-isothiocyanate (FITC ‘Isomer II’) |
| T-353 | Texas Red ® sulfonyl chloride |
| T-1905 | Texas Red ® sulfonyl chloride |
| T-10125 | Texas Red ®-X, STP ester, sodium salt |
| T-6134 | Texas Red ®-X, succinimidyl ester |
| T-20175 | Texas Red ®-X, succinimidyl ester |

Phospholipids having a head group that comprises a functional group, R^{x}, for coupling to a fluorescent dye can be prepared synthetically by conventional methods, can be obtained from natural sources or purchased commercially. Examples of such functional groups include amine, thiol, succinyl, glutaryl, and N-caproylamine. Non-limiting examples of reactive phospholipids are indicated in Table 4.

TABLE 4

| | | Avanti Catalog |
|---|---|---|
| Product Acyl Composition | M.W. | Number |
| Phosphatidylethanolamine 16:0 | 691.97 | 850705 |
| Phosphatidylethanolamine 18:1 | 744.05 | 850725 |
| N-Caproylamine-PE 16:0 | 805.13 | 870125 |
| N-Caproylamine-PE 18:1 | 857.21 | 870122 |
| N-Dodecanylamine-PE 16:0 | 889.29 | 870140 |
| N-Dodecanylamine-PE 18:1 | 941.37 | 870142 |
| Phosphatidylthio-ethanol 16:0 | 731.00 | 870160 |
| N-MCC-PE 16:0 | 928.24 | 780200 |
| N-MCC-PE 18:1 | 980.32 | 780201 |
| N-MPB-PE 16:0 | 955.20 | 870013 |
| N-MPB-PE 18:1 | 1,007.27 | 870012 |
| N-PDP-PE 16:0 | 911.22 | 870205 |
| N-PDP-PE 18:1 | 963.30 | 870202 |
| N-Succinyl-PE 16:0 | 814.03 | 870225 |
| N-Succinyl-PE 18:1 | 866.10 | 870222 |
| N-Glutaryl-PE 16:0 | 828.05 | 870245 |
| N-Glutaryl-PE 18:1 | 880.13 | 870242 |
| N-Dodecanyl-PE 16:0 | 926.24 | 870265 |
| N-Dodecanyl-PE 18:1 | 978.32 | 870262 |
| N-Biotinyl-PE 16:0 | 940.25 | 870285 |
| N-Biotinyl-PE 18:1 | 992.32 | 870282 |
| N-Biotinyl Cap-PE 16:0 | 1,053.40 | 870277 |
| N-Biotinyl Cap-PE 18:1 | 1,105.48 | 870273 |
| Phosphatidyl (Ethylene Glycol)16:0 | 714.94 | 870305 |
| Phosphatidyl (Ethylene Glycol)18:1 | 767.01 | 870302 |

where N-MCC-PE 16:0 refers to 1,2-Dipalmitoyl-sn-glycero-3-phosphoethanolamine-N-[4-(p-maleimidomethyl)cyclohexane-carboxamide]; 16:0 MPB PE refers to 1,2-Dipalmitoyl-sn-Glycero-3-Phosphoethanolamine-N-[4-(p-maleimidophenyl)butyramide] (sodium salt); and 16:0 PDP PE refers to 1,2-Dipalmitoyl-sn-Glycero-3-Phosphoethanolamine-N-[3-(2-pyridyldithio)propionate] (sodium salt).

Phospholipids can be prepared using conventional synthetic methods. The synthesis of phospholipids is described in Phospholipids Handbook, G. Cevc, ed., Marcel Dekker (1993), G Hermanson, Bioconjugate Techniques, Academic Press (1996) and Subramanian et al. ARKIVOC VII:116-125 (2002)). For example, phospholipids can be prepared from the reaction of a 3-substituted phosphoglycero compound with selected fatty acid anhydrides. Alternatively, phospholipids can be extracted from natural sources (e.g. egg yolk, brain, or plant sources) or can be purchased commercially (e.g. from Sigrna-Aldrich and Avanti Polar Lipids). Examples of suitable phosphoglycero compounds include glycero-3-phosphoethanolamine and glycerol-3-phosphoserine, either of which can be obtained commercially (e.g. from Sigma-Aldrich), or obtained by extraction from natural sources. Fatty acid anhydrides are prepared from fatty acids which in turn can be synthesized by conventional methods, extracted from natural sources, or purchased commercially.

Fluorescent dyes can also be attached to phospholipid head groups by the use of specific binding pair members as is known in the art (such as listed in U.S. Pat. Nos. 6,399,392 and 6,716,994). Examples of specific binding pairs include biotin/avidin, carbohydrate/lectin, DNA/cDNA, IgG/proteinA and ion/chelator.

A variety of fluorescently-labeled phospholipase substrates suitable for use in the instant methods are commercially available. Non-limiting examples of commercially available substrates include 1-Hexanoyl-2-[6-[(7-nitro-2-1,3-benzoxadiazol-4-yl)amino]hexanoyl]-sn-Glycero-3-Phosphocholine (catalog no. 810112, Avanti); 1,2-Dimyristoyl-sn-Glycero-3-Phosphoethanolamine-N-(Lissamine Rhodamine B Sulfonyl) (Ammonium Salt) (catalog no. 810157, Avanti); 1,2-Dioleoyl-sn-Glycero-3-Phosphoethanolamine-N-(5-dimethylamino-1-naphthalenesulfonyl) (catalog no. 790628, Avanti); 1,2-Dioleoyl-sn-Glycero-3-Phosphoethanolamine-N-(1-pyrenesulfonyl) (catalog no. 790627, Avanti); and 1,2-Dioleoyl-sn-Glycero-3-Phosphoethanolamine-N-(Carboxyfluorescein) (catalog no. 790547, Avanti). Other examples include Oregon Green® 488 1,2-dihexadecanoyl-sn-glycero-3- phosphoethanolamine (Oregon Green® 488 DHPE) (catalog no. 0-12650, Molecular Probes) and 1,2-dioleoyl-sn-glycero-3-phosphoethanolamine-N-(carboxyfluorescein) (catalog no. 790547, Avanti).

In the practice of the methods, the fluorescently-labeled phospholipase substrate is present in a lipid complex. Essentially any lipid complex can be used as long as the phospholipase substrate is retained in association with the lipid complex such that the substrate is quenched and is accessible for phospholipase-mediated cleavage. For example, the lipid complex can be a micelle or a liposome. Micelles form from single-chain and/or double-chain amphiphiles and comprise small structures in which surface polar head shield the nonpolar interior against water. Amphiphiles are molecules that contain two groups with different solubility. The hydrophilic group, often referred to as the polar head, is “water loving”, while the hydrophobic part, the so-called nonpolar tail, is “water hating”. Micelles can also form a variety of structures in aqueous media, including spherical, oblate, prolate, and cubic structures. Micelles can be formed from single chain amphiphiles, such as soaps and detergents, or from double chain amphiphiles. Any single chain amphiphile, double chain amphiphile or mixture of such amphiphiles, can be used in preparing micelles for use herein. Examples include fatty acid chains, such as saturated or unsaturated fatty acids, and are exemplified by those listed in Table 1 herein.

In some embodiments, the lipid complex can be a liposome. A liposome is a self-closed vesicle where one or several lipid membranes encapsulate part of the solvent. The composition and form of these lipid vesicles are analogous to that of cell membranes with hydrophilic polar groups directed inward and outward toward the aqueous media and hydrophobic fatty acids intercalated within the bilayer. Liposomes are formed when thin lipid films or lipid cakes are hydrated and stacks of liquid crystalline bilayers become fluid and swell and may be unilamellar and/or multilamellar. Unilamellar vesicles are typically classified as small (20 to 200 nm diameter) or large (approximately 1 micron). Large, multilamellar vesicles (LMV) are also formed. Multilamellar liposomes are classically described as having concentric bilayers, an “onion morphology”. A type of multilamellar liposome termed oligolamellar liposomes are typically described as multilamellar liposomes with increased aqueous space between bilayers or which have liposomes nested within bilayers in a nonconcentric fashion. Once these particles have formed, reducing the size of the particle requires energy input in the form of sonic energy (sonication) or mechanical energy (extrusion).

Liposomes useful in the present disclosure typically include amphiphiles having two nonpolar tails or other bulky structure such that the amphiphiles cannot be packed into micelles. Liposomes can be formed from any single type of amphiphile or from a mixture of amphiphiles. A liposome preparation can include one or more of phosphatidic acid, phosphatidylethanolamine, phosphatidylcholine, phosphatidylinositols, phosphatidylglycerol, sphingomylelin, cardiolipin, lecithin, phosphatidylserine, cephalin, cerebrosides, dicetylphosphate, steroids, terpenes, stearylamine, dodecylamine, hexadecylamine, acetylpalmitate, glycerol ricinoleate, hexadecyl stearate, isopropyl myristate, dioctadecylammonium bromide, amphoteric polymers, triethanolamine lauryl sulfate and cationic lipids, 1-alkyl-2-acyl-phosphoglycerides, and 1-alkyl-1-enyl-2-acyl-phosphoglycerides. Other amphiphiles useful in forming micelles include cationic lipids, such as described in Lasic (1997), pp. 81-86, examples of which include dioctadecyl dimethyl ammonium bromide/chloride (DODAB/C) and dioleoyloxy-3-(trimethylammonio)propane (DOTAP).

A wide variety of suitable lipids are commercially available (such as from Avanti Polar Lipids, Inc. Alabaster, Ala.). Liposome kits are commercially available (e.g. from Boehringer-Mannheim, ProMega, and Life Technologies (Gibco)). Non-limiting examples of suitable lipids include 1,2-dimyristoyl-sn-glycero-3-phosphate (Monosodium Salt) (DMPA•Na) (Avanti catalog no. 830845), 1,2-dimyristoyl-sn-glycero-3-phosphate (Monosodium Salt) (DOPS•Na) (Avanti catalog no. 830035), and 1,2-dioleoyl-3-trimethylammonium-propane (Chloride Salt) (DTOAP•Cl) (Avanti catalog no. 890890).

The liposomes can also include synthetic lipid compounds such as D-erythro (C-18) derivatives including sphingosine, ceramide derivatives, and sphinganine; glycosylated (C18) sphingosine and phospholipid derivatives; D-erythro (C17) derivatives; D-erythro (C20) derivatives; and L-threo (C18) derivatives, all of which are commercially available (Avanti).

Liposomes can include or be wholly formed from non-naturally occurring analogs of phospholipids that are resistant to lysis by certain phospholipases. In some embodiments of such analogs, the phosphate group is replaced by a phosphonate or phosphinate group (as described in U.S. Pat. No. 4,888,288). In addition, if the phospholipid normally includes an ester moiety (ester of a fatty acid), the ester linkage can be replaced with an ether linkage (e.g. as shown at position 31 and/or 33 in FIG. 1).

In some embodiments, liposomes which have been found to be useful in this disclosure incorporate phosphatidylethanolamine into the bilayer that can be formed into a vesicular structure. The liposome can be formed from mixtures of lipids such as phosphatidylcholine (PC) and phosphatidylethanolamine (PE). The phosphatidylcholine can range from about 50 to 95 mol % of the lipid content of the liposome, but can range from 60 to 90 mol %, while phosphatidylethanolamine can range from 2 to 20 mol %, but can range from about 4 to 12 mol % of the lipid content of the liposome.

Fluorescently labeled phospholipase substrates of the present disclosure can be prepared as described above and incorporated into a mixture of lipids prior to formation of the lipid complex. Alternatively, a lipid complex preparation can include lipids having reactive head groups, such phosphatidylenthanolamine, externally exposed, and which can be reacted with a fluorescent dye having a complementary reactive linking group as described hereinabove.

It will be appreciated that more than one fluorescent label can be used in preparing lipid complexes. Lipid complexes can be prepared with two (or more) different phospholipase substrates having different, spectrally resolvable labels. In and exemplary embodiment (FIG. 2), liposomes can be prepared with a first fluorescent label D1 (40) linked to a phospholipase substrate that is recognized by a first phospholipase, and a second fluorescent label D2 (42) linked to a second substrate that is recognized by a second phospholipase. This can allow the simultaneous determination of more than one phospholipase activity. For example, a first label can be linked to a PLC-specific substrate and a second label can be linked to a PLA2-specific substrate. The first and second labels have different fluorescent properties and can be detected independently.

It will be appreciated that certain phospholipases can recognize certain phospholipase substrates that are free in solution as well as labeled substrates that are incorporated into a lipid complex. For example, lipid complexes carrying labeled substrates as provided herein can be combined in solution with aqueously soluble phospholipase substrates, wherein such aqueously soluble substrates can be hydrolyzed by particular phosphorylases. For example, lipid complexes as described herein can be mixed with the aqueously soluble and non-fluorescent 4-methylumbelliferyl phosphocholine (catalog no. M-2885, Molecular Probes) which is cleaved by PLC, and not by PLD, to yield a blue fluorescent product.

Lipid complexes, such as micelles or liposomes, can be prepared using one or more of the amphiphilic compounds described above and including in the mixture one or more fluorescently-labeled phospholipase substrates as described herein. For example, liposomes can be prepared to incorporate one or more fluorescently labeled substrates. A labeled substrate can present in the liposomal membrane at any appropriate concentration. In some embodiments, the labeled substrate is present in the range of 1 mol % to 100 mol %. In some embodiments, the labeled substrate is present in the range of 30 mol % to 70 mol %. In some embodiments, the labeled substrate is present at 5 mol %, 10 mol %, 20 mol %, 40 mol %, 60 mol %, 80 mol % or 100 mol %. The substrate is typically present at a mol % concentration at which the fluorescent signal is quenched prior to the addition of phospholipase, as described herein. The exact composition of the lipid complex can be varied and a suitable composition determined empirically to obtain the desired results.

In an exemplary embodiment (FIG. 2), liposomes (or other lipid complex) can be prepared to include in their bilayer a fluorescent label D1 (40) linked to a phospholipase substrate that is recognized by a phospholipase and also a quenching moiety D2 (42) linked to a second phospholipid. The second phospholipid can be designed to be inert to phospholipase cleavage, as described below. The fluorescent signal of D1 is reduced due to quenching by D2, but the signal increases when D1 is cleaved from the substrate and can diffuse away from D2. Examples of suitable quenching moieties include FAM, TAMRA, DABCYL, Eclipse™ Dark Quenchers (Epoch Biosciences), NTB (Applied Biosystems), Black Hole Quenchers™ (Biosearch Technologies), Iowa Black 3.1™ (Integrated DNA Technologies), and QSY® 7 amine (Molecular Probes).

In an exemplary embodiment (FIG. 3), hydrophilic fluorescent dye “D3” (44) can be encapsulated within the liposome (or other lipid complex) which incorporates a phospholipase substrate coupled to label “D1” (46) in the liposomal bilayer. D3 can act as a tracer to assess the integrity of the liposome during preparation, storage or during a phospholipase assay. Certain phospholipases can cleave phospholipids at a position that leads to loss of integrity of the liposomal bilayer, and would thus release encapsulated label. Such a dual label system can be used to differentiate the phospholipase being tested. PLD, for example, would not be expected to lead to release of internal label, but would instead release label to the external aqueous environment. PLA2 activity, on the other hand, causes cleavage of fatty acid from the lipid, resulting in loss of integrity of the liposomal membrane. Thus, a liposome prepared with fluorescent label D3 encapsulated within the liposome, and also with a substrate coupled to a label D1 having fluorescent properties differing from label D3, could be used to screen for PLD and PLA2 activities simultaneously. In some embodiments, a mixture of different aqueously soluble fluorescent dyes, having different spectral properties, can be loaded within a liposome. In some embodiments, D3 can comprise an aqueously soluble quenching dye, which quenches the fluorescence of D1 thereby enhancing the fluorescent signal of D1 upon phospholipase-mediated release of D1. Suitable dyes for use as internal labels and as quenching agents include xanthene and rhodol derivatives, such as described herein and in U.S. Pat. Nos. 5,227,487 and 6,399,392. The choice of quencher D3 will depend on the identity of label D1. In some embodiments in which fluorescent compounds, or quenching agents are encapsulated into liposomes, conventional methods can be used for loading, such as reverse phase methods and sonication (e.g. as described by Lasic (1997) p. 93 and in U.S. Pat. No. 4,888,288).

In some embodiments, lipophilic fluorescent dyes can be embedded non-covalently within the lipid phase of a lipid complex, to facilitate quenching or to act as a tracer to detect the integrity of the lipid complex during a phospholipase assay. Without wishing to be bound by theory, it is believed that such dyes are embedded within the lipid membrane by van der Waals forces.

Properties of liposomes can vary depending on the composition (cationic, anionic, neutral lipid species), however, the same preparation method can be used for all lipid vesicles regardless of composition. The general elements of the procedure involve preparation of the lipid for hydration, hydration with agitation, and sizing to a homogeneous distribution of vesicles.

Lipid complexes can be prepared using conventional methods (such as described in Lasic, Liposomes in Gene Delivery, CRC Press, New York pp. 67-112 (1997), Ann. Rev. Biophys. Bioeng. 9:467-508 (1980); U.S. Pat. Nos. 4,229,360, 4,241,046 4,235,871, 6,458,381 and 6,534,018). For example, when preparing liposomes with mixed lipid composition, the lipids can first be dissolved and mixed in an organic solvent to assure a homogeneous mixture of lipids. Usually this process is carried out using chloroform or chloroform:methanol mixtures. The intent is to obtain a clear lipid solution for complete mixing of lipids. Typically lipid solutions are prepared at 10-20 mg lipid/ml organic solvent, although higher concentrations may be used if the lipid solubility and mixing are acceptable. Once the lipids are thoroughly mixed in the organic solvent, the solvent is removed to yield a lipid film. For small volumes of organic solvent (<1 mL), the solvent may be evaporated using a dry nitrogen or argon stream in a fume hood. For larger volumes, the organic solvent can be removed by rotary evaporation yielding a thin lipid film on the sides of a round bottom flask. The lipid film is thoroughly dried to remove residual organic solvent by placing the vial or flask on a vacuum pump overnight. If the use of chloroform is objectionable, an alternative is to dissolve the lipid(s) in tertiary butanol or cyclohexane. The lipid solution is transferred to containers and frozen by placing the containers on a block of dry ice or swirling the container in a dry ice-acetone or alcohol (ethanol or methanol) bath. Care should be taken when using the bath procedure that the container can withstand sudden temperature changes without cracking. After freezing completely, the frozen lipid cake is placed on a vacuum pump and lyophilized until dry (1-3 days depending on volume). The thickness of the lipid cake should be no more than the diameter of the container being used for lyophilization. Dry lipid films or cakes can be removed from the vacuum pump, the container close tightly and taped, and stored frozen until ready to hydrate.

Hydration of the dry lipid film/cake is accomplished simply by adding an aqueous medium to the container of dry lipid and agitating. The temperature of the hydrating medium should be above the gel-liquid crystal transition temperature (Tc) of the lipid with the highest Tc before adding to the dry lipid. After addition of the hydrating medium, the lipid suspension should be maintained above the Tc during the hydration period. For high transition lipids, this is easily accomplished by transferring the lipid suspension to a round bottom flask and placing the flask on a rotary evaporation system without a vacuum. Spinning the round bottom flask in the warm water bath maintained at a temperature above the Tc of the lipid suspension allows the lipid to hydrate in its fluid phase with adequate agitation. Hydration time may differ slightly among lipid species and structure, however, a hydration time of 1 hour with vigorous shaking, mixing, or stirring is highly recommended. It is also believed that allowing the vesicle suspension to stand overnight (aging) prior to downsizing makes the sizing process easier and improves the homogeneity of the size distribution. Aging is not recommended for high transition lipids as lipid hydrolysis increases with elevated temperatures. The hydration medium is generally determined by the application of the lipid vesicles. Suitable hydration media include distilled water, buffer solutions, saline, and nonelectrolytes such as sugar solutions. During hydration some lipids form complexes unique to their structure. Highly charged lipids have been observed to form a viscous gel when hydrated with low ionic strength solutions. The problem can be alleviated by addition of salt or by downsizing the lipid suspension. The product of hydration is a large, multilamellar vesicle (LMV) analogous in structure to an onion, with each lipid bilayer separated by a water layer. The spacing between lipid layers is dictated by composition with poly hydrating layers being closer together than highly charged layers which separate based on electrostatic repulsion. Once a stable, hydrated LMV suspension has been produced, the particles can be downsized by a variety of techniques, including sonication or extrusion.

Disruption of LMV suspensions using sonic energy (sonication) typically produces small, unilamellar vesicles (SUV) with diameters in the range of 15-50 nm. Instrumentation for preparation of sonicated particles are bath, probe tip and cup-horn sonicators. Sonication of an LMV dispersion is accomplished by placing a test tube containing the suspension in a bath sonicator (or placing the tip of the sonicator in the test tube) and sonicating for 5-10 minutes above the Tc of the lipid. The lipid suspension should begin to clarify to yield a slightly hazy transparent solution. The haze is due to light scattering induced by residual large particles remaining in the suspension. These particles can be removed by centrifugation to yield a clear suspension of SUV. Mean size and distribution is influenced by composition and concentration, temperature, sonication time and power, volume, and sonicator tuning.

An alternative method for sizing is extrusion. Lipid extrusion is a technique in which a lipid suspension is forced through a polycarbonate filter with a defined pore size to yield particles having a diameter near the pore size of the filter used. Prior to extrusion through the final pore size, LMV suspensions can be disrupted either by several freeze-thaw cycles or by prefiltering the suspension through a larger pore size (typically 0.2 μm-1.0 μm). This method helps prevent the membranes from fouling and improves the homogeneity of the size distribution of the final suspension. As with all procedures for downsizing LMV dispersions, the extrusion should be done at a temperature above the Tc of the lipid. Attempts to extrude below the Tc will be unsuccessful as the membrane has a tendency to foul with rigid membranes which cannot pass through the pores. Extrusion through filters with 100 nm pores typically yields large, unilamellar vesicles (LUV) with a mean diameter of 120-140 nm. Mean particle size also depends on lipid composition and is quite reproducible from batch to batch.

Liposomes can include cholesterol. Cholesterol intercalates within the phosphatidylcholine bilayer with very little change in area by occupying the regions created by the bulky phosphatidylcholine headgroups. This increases the packing density and structural stability of the bilayer (New, R.R.C., 1990 In New, R.R.C. (ed): Liposomes: a practical approach, Oxford University Press, New York, pp 19-21). The concentration of cholesterol in liposomes can be in the range, for example, of about 5 to about 60 mol %, although higher or lower concentrations can be used.

The composition of the lipid mixture can be selected based an a variety of factors including cost, transition temperature of the lipids, stability during storage, and stability of the liposomes under the reaction conditions and in the presence of the phospholipase activity being characterized.

Preparations of lipid complexes can include stabilizing agents including antioxidants, such as α-tocopherol and chelators. Other agents include ascorbic acid, cysteine, monothioglycerol, sodium bisulfite, sodium metabisulfite, gentisic acid and inositol. Lipid complexes can be lyophilized for storage and/or for use in kits as described hereinbelow.

In some embodiments, the method includes detecting the fluorescence of a fluorescently-labeled phospholipase substrate moiety as a function of time, wherein an increase in fluorescence as a function of time correlates with the presence of the phospholipase in the sample. While the basis for increased fluorescence may not be certain, and without wishing to be bound by theory, it is contemplated that the fluorescent moieties of the substrates quench each other due to their close proximity within the lipid complex. The product of the phospholipase reaction, however, is released from the close proximity and the label from the product fluoresces brightly since it remains free from other fluorescent moieties in the lipid complex. This is shown schematically in FIG. 4 in which the fluorescent label 48 is attached to a phospholipase substrate (not shown) incorporated into the membrane 50 of a lipid complex. The released label is indicated at 52.

Detection of an increase in fluorescent signal in the mixture indicates the presence of a phospholipase in the sample. The phospholipase to be detected can be any phospholipase known in the art. Also, the phospholipase can be a phospholipase candidate, and the method is used to confirm and/or characterize the phospholipase activity of the candidate. A variety of phospholipases have been identified (e.g., Methods in Enzymology, E. A. Dennis, ed. Vol. 197, Academic Press (1991)). In some cases, phospholipases are highly specific for only one or a few substrates. Accordingly, the phospholipase substrates of the present disclosure can be designed to detect particular phospholipase by suitable selection of the phospholipase substrate.

The present disclosure contemplates not only detecting target phospholipases, but also methods involving: (1) screening for and/or quantifying phospholipase activity in a sample, (2) determining apparent Km of a phospholipase or phospholipase mixture with respect to selected substrates, (3) detecting, screening for, and/or characterizing substrates of phospholipase, and (4) detecting, screening for, and/or characterizing inhibitors, activators, and/or modulators of phospholipase activity. For example, in screening for phospholipase activity, a sample that contains, or may contain, a particular phospholipase activity is mixed with a substrate as described herein, and the fluorescence is measured to determine whether an increase in fluorescence has occurred. Screening may be performed on numerous samples simultaneously in a multi-well or multi-reaction plate or device to increase the rate of throughput.

A fluorescently-labeled phospholipase substrate can be designed to be cleaved by a particular phospholipase or a group of phospholipases, or it can be designed to determine substrate specificity and other catalytic features, such as determining an apparent Km, or apparent Ki. Naturally occurring phospholipids and sphingolipids can be used in preparing fluorescently labeled phospholipase substrates and include diacyl phospholipids, sphingolipids, vinyl ether phospholipids and alky ether phospholipids. An ether linkage between a hydrophobic tail group and the three carbon lipid backbone can make the substrate resistant to PLA1 or PLA2 activity, for example, but still reactive with PLC or PLD. In some embodiments, a fluorescently labeled phospholipase cleavage substrate having only a single hydrophobic tail group, can be used in the present methods. A fluorescently labeled phospholipase cleavage substrate can be formed from non-naturally occurring analogs of phospholipids that are resistant to lysis by certain phospholipases. In some embodiments of such analogs, the phosphate group is replaced by a phosphonate or phosphinate group (as described in U.S. Pat. No. 4,888,288). In addition, if the phospholipid normally includes an ester moiety (ester of a fatty acid), the ester linkage can be replaced with an ether linkage at position C1 or C2, thus rendering the substrate resistant to PLA1 or PLA2 cleavage.

In one aspect, the disclosure provides a method for screening for modulators of phospholipase activity. A modulator can be an inhibitor or an activator of phospholipase activity. In some embodiments, a selected modulator can be added concurrently with a fluorescently labeled phospholipase substrate, and the activity detected continuously, or non-continuously such as at selected time points. In some embodiments, a modulator can be added in a “pre-incubation” prior to adding substrate. When screening a variety of modulators, an arbitrary criteria can be selected in order to identify a candidate compound or substance as a modulator. For example, the rate of cleavage of a known phospholipase substrate can be determined under defined assay conditions. A candidate compound can be added to a separate incubation, and a 10% or greater increase in the rate of cleavage would classify the candidate compound as an activator. In another example, a 10% or greater decrease in activity would classify the candidate compound as an inhibitor. In some embodiments, replicates of assays can be run in the presence and in the absence of a candidate modulator compound, and the results analyzed using standard statistical methods. Candidate compounds can be compared to standard modulators consisting of known inhibitors or activators, such as described hereinabove. Prior to evaluating modulators, assays can be optimized to determine a suitable concentration of fluorescently labeled phospholipase substrate. A suitable range of time, such one providing a linear time course can be used. Standard curves can be prepared showing the reaction rate at various concentrations of labeled phospholipase substrate and other curves can be prepared showing the effect of various concentrations of standard modulators or of candidate modulators.

Various inhibitors can be used to selectively inhibit phospholipase activities. For example, D 609 (tricyclodecan-9-yl xanthogenate) (catalog no T-6615, Molecular Probes) is a selective inhibitor of PLC. Various peptides can be used as inhibitors (disclosed in J. Biol. Chem., 267, 21844-21849 (1992)). A number of small molecules have been employed as inhibitors of calcium-independent phospholipase A2 (PLA2 group VI), including: arachidonyl trifluoromethyl ketone, arachidonyl tricarbonyl and methyl arachidonyl fluorophosphonate which function as transition state analogues and bromoenol lactone which acts an irreversible mechanism-based inhibitor (Balsinde et al., J. Biol. Chem. 272:16069-16072 (1997)). Other inhibitors of PLA2 include p-bromophenacyl bromide (Sigma, Cat. #B2006) and dehydroabietylamine acetate (Sigma-Aldrich, Cat. #29,356-3). Antibodies specific for various phospholipases can be used, and are commercially available (Sigma, Molecular Probes). An example is rabbit anti-phospho-phospholipase C g-1 (pTyr⁷⁸³) antibody (Sigma, Cat. #P6111).

Activators of phospholipase activity can also be used. An exemplary activator of PLA2 is a 21 amino acid peptide as available from Sigma-Aldrich (cat. #. F1153).

A variety of phospholipases, such as PLA1s, PLA2s, PLCs and PLDs can be assayed using the present methods. For example, various PLA2 isozymes, including secretory (sPLA2), cytosolic (cPLA2), and Ca²⁺-independent PLA2s (as described in Murakami et al., Crit. Rev. Immunol. 17:225-83 (1977)), can be assayed. At least 10 PLC isozymes have been described (U.S. Pat. No. 6,534,301, Lee et al. Curr. Opin. Cell. Biol. 2:183-9 (1995), Rhee Science 244:546-50 (1989)) and various PLD isozymes have been described (Liscovitch, Biochim. Biophys. Acta 1439:245-263 (1999)) all of which are amenable to analysis using the present method.

Phospholipase preparations can be obtained commercially from a variety of sources (e.g. Sigma-Aldrich, Avanti, Molecular Probes) for use as controls or as standards. Non-limiting examples include PLA2 from bovine pancreas (catalog no. P9279, Sigma), PLC from Bacillus cereus (Cat. #P7147, Sigma), phospholipase B from Vibrio sp (Cat. #P8914, Sigma) and PLD from Arachis hypogaea (Cat. #P0515, Sigma).

The methods as disclosed herein can also be used to screen mutant forms of phospholipases. Mutant forms of phospholipase can be obtained from naturally occurring sources, or can be obtained by conventional site-directed mutagenesis methods or “gene evolution” methods (e.g. as taught in U.S. Pat. Nos. 5,605,793 and 6,335,160 and PCT publication WO 00/09755).

In practice, a lipid complex comprising a fluorescently labeled phospholipase cleavage substrate is mixed with a sample containing an enzyme that is to be detected or that is being used to screen for, detect or characterize a compound for substrate, inhibitor, activator, or modulator activity. The phospholipase reaction mixture typically includes a buffer, such as a buffer described in the “Biological Buffers” section of the 2003 Sigma-Aldrich Catalog. Exemplary buffers include sodium phosphate, sodium acetate, MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the activity of the phospholipase and/or the fluorescent properties of the dye to be detected. For example, the pH can be from 2 to 12, from 4 to 11, or from 6 to 10. The reaction mixture also contains any necessary cofactors (e.g., Ca²⁺ ion). The concentration of lipid complexes in the incubation can range from about 0.001 μM to about 1 mM (based on total lipid). The concentration of phospholipase in the reaction can be in the range of about 0.001 μg/ml to about 100 μg/ml.

The phospholipase assay reaction mixture typically does not require the presence of cofactors such as albumin. In general, it is desirable to avoid high concentrations of components in the reaction mixture that can adversely affect the fluorescence properties of the reaction product, or that can interfere with the analysis of modulators. Johnson et al. (J. Biol. Chem. 255:3466-3471 (1980)) describe a lipoprotein lipase assay using dansyl phosphatidylethanolamine-labeled very low density lipoproteins (DPE-VLDL). The fluorescence changes that occurred with the cleavage of DPE-VLDL by lipoprotein lipase were dependent on the presence of albumin (0.4% BSA) in the assay. Dansyl does not fluoresce in aqueous medium. The fluorescence changes described by Johnson et al. were explained by a mechanism involving the hydrolysis of DPE to lyso-DPE and the subsequent binding of lyso-DPE to albumin. In contrast, the present methods do not require the use of albumin in order to detect fluorescence changes associated with substrate cleavage. In some embodiments, fluorescent dyes used in making the labeled substrates of the present disclosure are capable of fluorescing in aqueous medium and do not require the presence of co-factors, such as albumin, in order to fluoresce. The dyes can be hydrophilic or hydrophobic.

The mol % concentration of fluorescently labeled phospholipase cleavage substrate in the lipid complex can be selected to obtain a useful dynamic range for the assay. In some embodiments, Applicant has observed that the lower the mol % of the labeled substrate in the lipid mixture used to make liposomes, the lower the degree of quenching observed. In order to select an appropriate concentration of labeled substrate, the concentration of fluorescently labeled phospholipase cleavage substrate can be varied. For example, a series of liposomes, or other lipid complexes, can be prepared having different mol % of labeled substrate. A mol % of substrate can be selected that provides a readily detectable signal and which gives a suitable dynamic range. An example of a selection procedure is provided in Example 6. The concentration of one or more lipids in the liposomal preparation can also be varied, and can affect the degree of quenching and the dynamic range observed.

In the methods described herein, the fluorescence signal can be detected using conventional methods and instruments. In some embodiments, a multiwavelength fluorescence detector can be utilized. The detector can be used to excite the detectable labels at one wavelength and detect emissions as multiple wavelengths, or excite at multiple wavelengths and detect at one emission wavelength. Alternatively, the sample can be excited using “zero-order” excitation in which the full spectrum of light (e.g., from xenon lamp) illuminates the cuvette. Each label can absorb at its characteristic wavelength of light and then emit maximum fluorescence. The multiple emission signals can be detected independently. Preferably, a suitable detector can be programmed to detect more than one excitation emission wavelength substantially simultaneously, such as that commercially available under the trade designation HP1100 (G1321A), from Hewlett Packard, Wilmington, Del. Thus, the labeled phospholipase substrates can be detected at programmed emission wavelengths at various intervals during a reaction.

Detection of fluorescent signal can be performed in any appropriate way. Advantageously, substrates as described herein can be used in a continuous assay, in real time, to allow the user to rapidly determine whether phospholipase activity is present in the sample, and optionally, the amount or specific activity of the phospholipase. The fluorescent signal is measured from at least two different time points, usually until an initial velocity (rate) can be determined. The signal can be detected continuously or at several selected time points. In some embodiments, the fluorescent signal can be measured in an end-point in which a signal is measured after a certain amount of time, and the signal is compared against a control signal (before start of the reaction), threshold signal, or standard curve.

The sample to be tested for phospholipase activity may be any suitable sample selected by the user. The sample may be naturally occurring or man-made. For example, the sample may be a blood sample, tissue sample, cell sample, buccal sample, skin sample, urine sample, water sample, or soil sample. The sample can be from a living organism, such as a eukaryote, prokaryote, mammal, human, yeast, or bacterium. More specifically, samples of phospholipases can be obtained from a variety of sources as indicated herein above. These include bacterial sources (e.g. E. coli), guinea pig pancreas, rat kidney, liver and uterus, PLA2 from cobra venom, PLB from Penicillin notatum. The sample may be processed prior to contact with a substrate as described herein by any method known in the art (e.g. see Dennin (1991)). For example, the sample be subject to a precipitation step, column chromatography step, heat step, etc. In some cases, the sample is a purified phospholipase that is used to screen for or characterize a phospholipase substrate, inhibitor, activator, or modulator. If the sample contains more than one class of phospholipase, so that the activity of one may interfere with the activity of the other, then an inactivating agent (e.g., an active site directed an irreversible inhibitor) can be added to the sample to inactivate whichever activity is not desired.

Another aspect the disclosure provides kits for performing methods as described herein. In some embodiments, the kit comprises at least one fluorescently labeled phospholipase cleavage substrate for detecting a target phospholipase, and a buffer for preparing a reaction mixture that facilitates the phospholipase reaction. The phospholipase substrate may be provided as part of a lipid complex preparation, such as a lyophilized preparation, provided in a container. The buffer may be provided in a container in dry form or liquid form. The choice of a particular buffer may depend on various factors, such as the pH optimum for the phospholipase to be detected, the solubility and fluorescence properties of the fluorescent moiety in the substrate, and the pH of the sample from which the target phospholipase is obtained. The buffer is usually added to the reaction mixture in an amount sufficient to produce a particular pH in the mixture. In some embodiments, the buffer is provided as a stock solution having a pre-selected pH and buffer concentration. Upon mixture with the sample, the buffer produces a final pH that is suitable for the enzyme assay, as discussed above. The pH of the reaction mixture may also be titrated with acid or base to reach a final, desired pH. The kit may additionally include other components that are beneficial to enzyme activity, such as salts (e.g., KCl, NaCl, or NaOAc), metal salts (e.g., Ca₂₊ salts such as CaCl₂, MgCl₂, MnCl₂, ZnCl₂ and/or other components that may be useful for a particular phospholipase. These other components can be provided separately from each other, such as in separate containers, or mixed together in dry or liquid form. In some embodiments, a kit can also include one or more of a lipid complex preparation including a fluorescence quenching compound anchored within the complex; an aqueously soluble quenching compound; lipid complex preparation including an aqueously soluble fluorescence quenching compound within the complex; a selected phospholipase inhibitor; a selected phospholipase activator; and a phospholipase enzyme standard. In some embodiments, kits may comprise the fluorescently labeled phospholipase substrate in combination with instructions for use setting forth any of the above-described methods.

Unless defined otherwise, all technical and scientific terms used herein have the same meaning as commonly understood by one of ordinary skill in the art to which this disclosure belongs. Unless mentioned otherwise the techniques employed or contemplated herein are standard methodologies well known to one of ordinary skill in the art. The materials, methods and examples are illustrative only and not limiting.

All numerical ranges in this specification are intended to be inclusive of their upper and lower limits.

Other features of the disclosure will be come apparent in the course of the following descriptions of exemplary embodiments which are given for illustration of the disclosure and are not intended to be limiting thereof.

### 7. EXAMPLES

### 7.1 Example 1

### Preparation of Fluorescently-Labeled Substrates and Cleavage by PLC

This example describes the preparation of fluorescently-labeled phospholipase substrates, and their cleavage by phospholipase. 1,2-dioleoyl-sn-glycero-3-phosphoethanolamine-N-(Oregon Green® 488, 5-isomer) (compound 60) (FIG. 5) was prepared by the following procedure. Oregon Green 488 carboxylic acid, succinimidyl ester 5-isomer 62 (25 mg, 49 μmol, Molecular Probes, Cat. #O-6147) was dissolved in dry DMF (1 ml) and added to 1,2-dimyristoyl-sn-glycero-3-phosphoethanolamine 64 (DMPE, Avanti Polar Lipids, Cat. #850745, 24 mg, 33 μmol) dissolved in dicholoromethane (1 ml) with added triethylamine (46 μl, 330 μmol). Oregon Green 488 has the following formula: 2,5-Pyrrolidinedione, 1-[[(2′,7′-difluoro-3′, 6′-dihydroxy-3-oxospiro[isobenzofuran-1(3H),9′-[9H]xanthen]-6-yl)carbonyl]oxyl]-. After 15 min the solvent was evaporated and the residue was dissolved in aqueous triethylammonium acetate buffer (TEAA, 20 ml, 2 M). The crude product was purified by reverse phase C18 HPLC eluting with a mixture of methanol and 100 mM TEAA (90:10 to 95:5). Pure fractions were combined, concentrated and desalted with a short plug of C18 reverse phase media to afford a yellow solid (22 mg, 20 μmol, 38%).

Small unilamellar vesicles (SUVs) composed solely of compound 60 were prepared by adding a concentrated solution of compound 60 in methanol (100 μl, 10 mM) drop-wise to a rapidly stirred aqueous solution of 20 mM HEPES/1 mM CaCl₂ buffer (100 ml, pH 7.5). It is well established in the literature that polydisperse SUVs form instantly upon addition of lipid in a small volume of ethanol to a large volume of rapidly stirred aqueous buffer (see Subroto et al. Banerjee in Methods in Molecular Biology: Liposome Methods and Protocols, Ed. by Basu et al., Humana Press, vol. 199, chapter 1, p 6 (2002)). The stock solution of compound 60 (10 μM) was further diluted with 20 mM HEPES/1 mM CaCl₂ to give the desired concentrations for the enzyme assay (0.2, 0.25, 0.3, 0.35, 0.4, 0.45, 0.5, 1.0 μM).

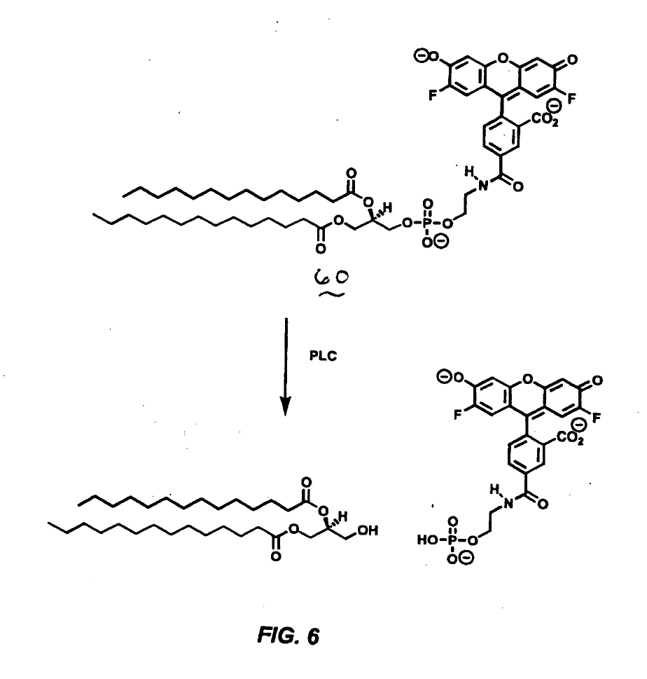

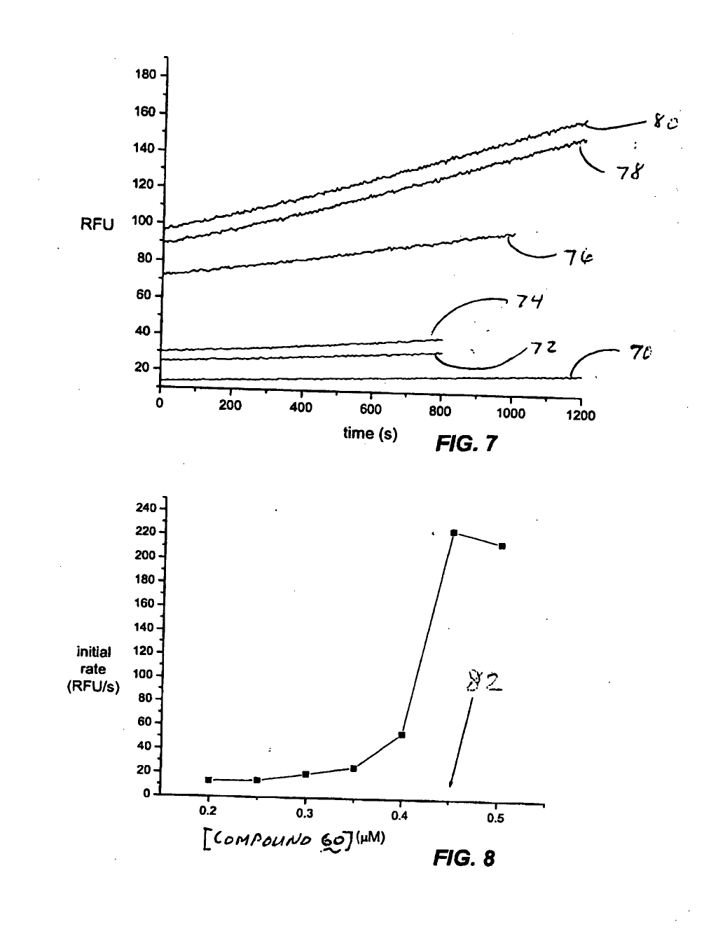

In order to detect cleavage of compound 60 (FIG. 6), a solution of compound 60 (3 ml, 1 μM) was added to a 4 ml disposable fluorescence cuvette. The initial fluorescence was measured on a Perkin-Elmer LS50 with λex/λem=500/520 nm and ex/em spectral slits set at 2.5/2.5 nm. A solution of PLC from Bacillus cereus (5 μl, 1 unit/ml, 0.9 μg protein/ml, Sigma, Cat. #P7147) was added and the change in fluorescence was detected every second for 1200 s (FIG. 7) at various initial concentrations of compound 60 as shown at 70, 72, 74, 76, 78, and 80 which correspond to the following concentrations (μM): 0.25, 0.30, 0.35, 0.40, 0.45, and 0.50, respectively. The temperature was held constant at 25° C. by a circulating water heater. The initial reaction rate was calculated by linear least squares analysis (Origin 6.0 program from OriginLab Co.) of the steady state portion of the data. The plot of initial rate in relative fluorescence units per second (RFU/s) versus initial concentration of compound 60 showed little change until 400 nM at which point the rate dramatically increased (FIG. 8). This behavior is typical of interfacial enzymes which have much higher Kₘ values for vesicles than monomers. The apparent critical micelle concentration (CMC) (arrow 82) of compound 60 is, therefore, 400 nM. The greater the net charge on the surfactant head group the greater the coulombic repulsion and, consequently, the greater the CMC (see Rosen, “Surfactants and Interfacial Phenomena” 2nd Ed., John Wiley and Sons, chapter 3, page 133 (1989)). DMPC has zero net charge and a CMC of about 4 nM while DMPS has negative one net charge and a CMC of about 60 nM (see Cevc, Phospholipids Handbook, Marcel Dekker, (1993) and Adrian et al. “Comparisons of solubilities predicted by the protrusion model with experimental critical micelle concentration estimates from the equations of Cevc and Marsh, Langmuir, 8:1502 (1992)). Without wishing to be bound by theory, it is believed that compound 60, with negative three net charge, would have a CMC of about 400 nM. (For a reference on determination of short-chain phosphatidylcholine CMC values by detecting PLC activity see El -Sayed et al., Sensitivity of phospholipase C activity to phosphatidylcholine structural modifications, Biochim. Biophys. Acta, 837:325 (1985)). Lamellar vesicles can be considered a type of micelle and, therefore, the term CMC also applies to SUV forming phospholipids (see Rosen in “Surfactants and Interfacial Phenomena”, 2nd Edition, John Wiley and Sons, page 112 (1989)).

A Clostridium perfringens PLC preparation obtained from Sigma-Aldrich (Cat. #P7633) was tested for cleavage of compound 60 and no activity was observed. The batch of enzyme was possibly inactivated during storage and/or shipment.

1,2-dimyristoyl-sn-glycero-3-phosphoethanolamine-N-(Oregon Green 500, 5-isomer) (compound 84) (FIG. 9) was prepared as follows. Oregon Green 500 carboxylic acid, succinimidyl ester (5-isomer) (25 mg, 49 μmol, Molecular Probes Cat. #O-6136) 86 was dissolved in dry DMF (1 ml) and added to 1,2-dimyristoyl-sn-glycero-3-phosphoethanolamine 64 (DMPE, Avanti Polar Lipids Cat. #850745, 24 mg, 33 μmol) dissolved in dicholoromethane (1 ml) with added triethylamine (46 μl, 330 μmol). After 15 min the solvent was evaporated and the residue was dissolved in aqueous triethylammonium acetate buffer (TEAA, 20 ml, 2 M). The crude product was purified by reverse phase C18 HPLC eluting with a mixture of methanol and 100 mM TEAA (90:10). Pure fractions were combined, concentrated and desalted with a short plug of C18 reverse phase media to afford a yellow solid (17 mg, 13 μmol, 38%).

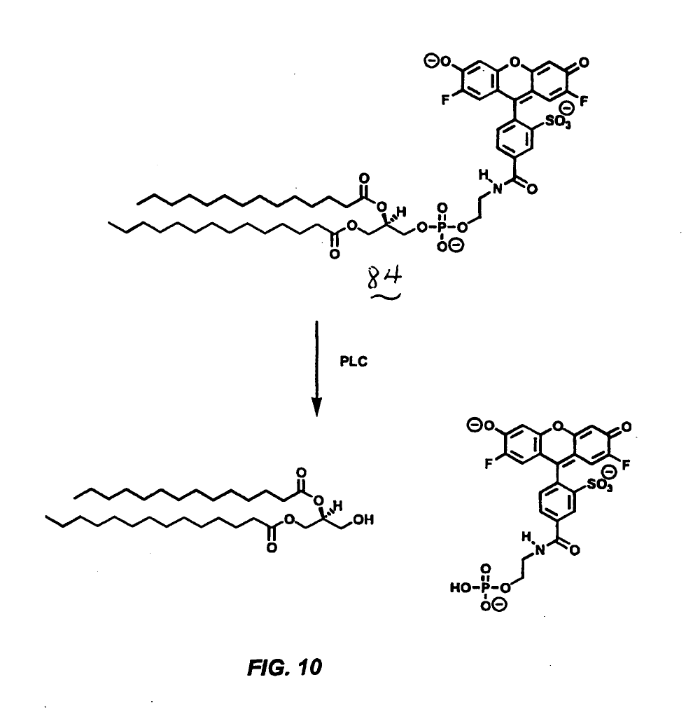

Cleavage of compound 84 by PLC (FIG. 10) and analysis of the products by HPLC and MS were carried out by the following procedures. A solution of compound 84 in methanol (0.1 ml, 10 mM) was added drop-wise to rapidly stirred aqueous 20 mM HEPES/1 mM CaCl₂ buffer (100 ml, pH 7.5). A solution of PLC (5 μl, 10 units/ml, 9 μg protein/ml) in 20 mM HEPES/1 mM CaCl₂ buffer was added to the stock solution of compound 84 (1 ml, 10 μM). Conversion of compound 84 to product was detected by reverse phase HPLC (methanol/100 mM TEAA, 80:20 to 90:10, 5 min). After about 30 min all of compound 84 had been converted to a polar product with the same absorption spectra (diode array detector). Electrospray mass spectral analysis of the reaction mixture supported the structural assignments (data not shown).

### 7.2 Example 2

### Effect of Inhibitor on PLC Substrate Cleavage

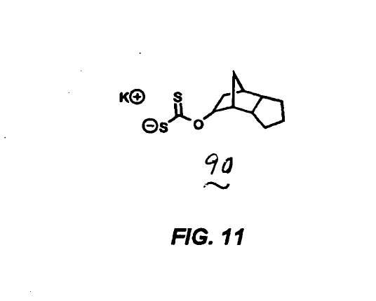

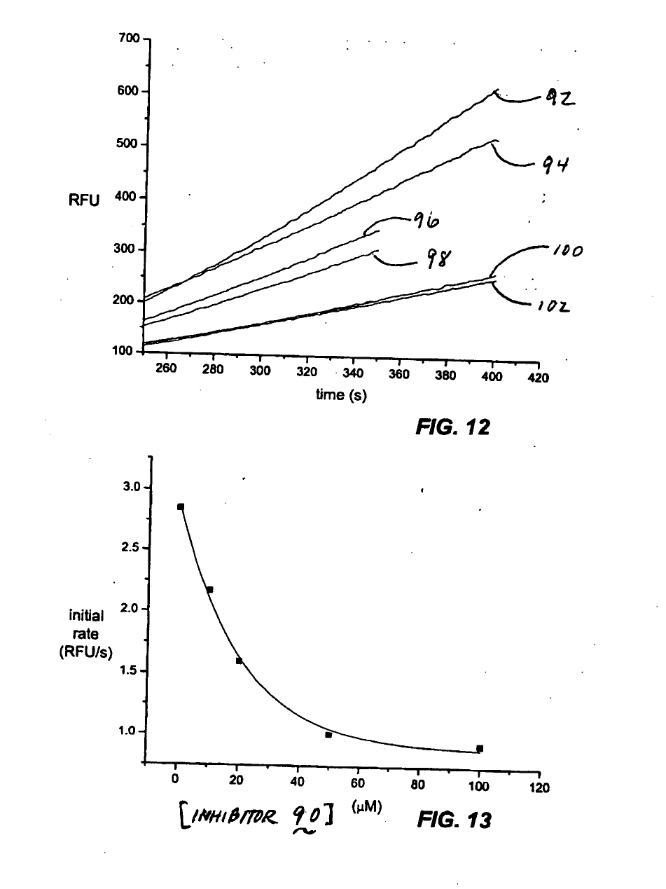

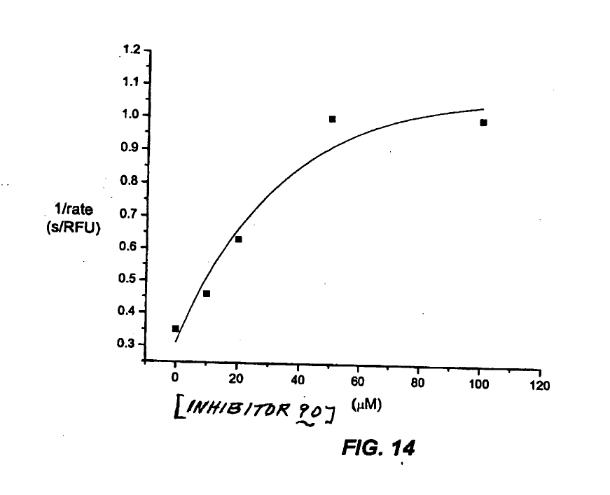

In order to determine the effect of inhibitor 90 on PLC-catalyzed cleavage of compound 60, the PLC cleavage reaction described in Example 1 was repeated with the concentration of compound 60 fixed at 0.5 μM and with various amounts of inhibitor 90 (tricyclodecan-9-yl-xanthogenate K, Calbiochem, Cat. #251400; see U.S. Pat. No. 4,602,037) added (FIG. 11). The raw fluorescence data as a function of time is shown in FIG. 12 and the plot of the initial rate versus concentration of inhibitor 90 is shown in FIG. 13. In FIG. 12, the concentrations of inhibitor 90 (μM) were 0, 10, 20, 30, 50 and 100, in the curves shown at 92, 94, 96, 98, 100 and 102, respectively. The IC₅₀ value was visually approximated to be 20 μM which is consistent with the Kᵢ values of 6 μM and 13-17 μM reported in the literature (E. Amtmann, “The antiviral antitumoral xanthate D609 is a competitive inhibitor of phosphatidylcholine-specific phospholipase C,” Drugs under Experimental and Clinical Research, 22:287-294 (1996); and A. Gonzalez-Roura, et al., “Synthesis and phospholipase C inhibitory activity of D609 diastereomers,” Lipids, 37:401-406 (2002)). The Dixon plot suggested that inhibitor 90 was acting as a partial inhibitor of PLC (FIG. 14).

### 7.3 Example 3

### Preparation of SWV's Composed of Compound 60

In this example, SUV's composed solely of compound 60 were prepared, and substrate cleavage by PLA2 was assessed. A solution of compound 60 in methanol (0.1 ml, 10 mM) was added drop-wise to rapidly stirred aqueous 20 mM TRIS/1 mM CaCl₂ buffer (100 ml, pH 8.0). This stock solution of SUV's composed of compound 60 (10 μM) was further diluted with 20 mM TRIS/1 mM CaCl₂ to give the desired concentrations for the enzyme assay (0.05, 0.1, 0.2, 0.3 μM).

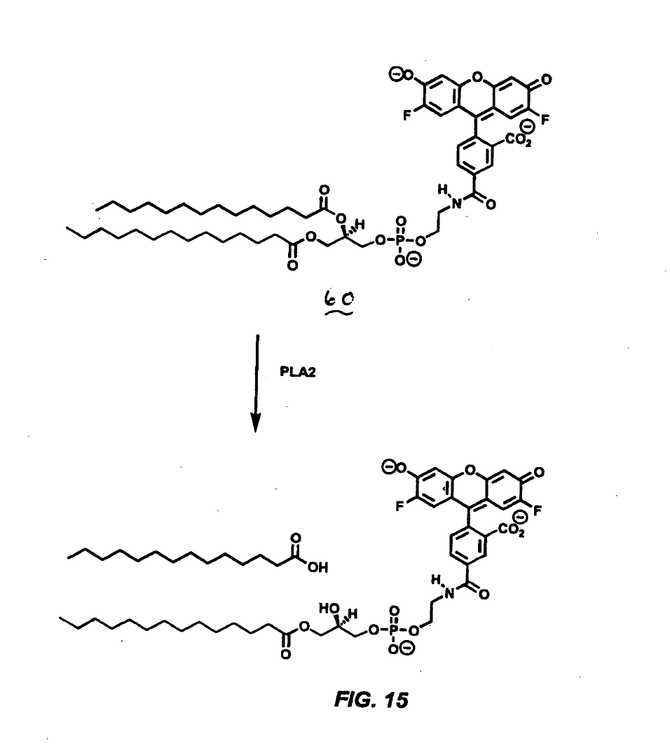

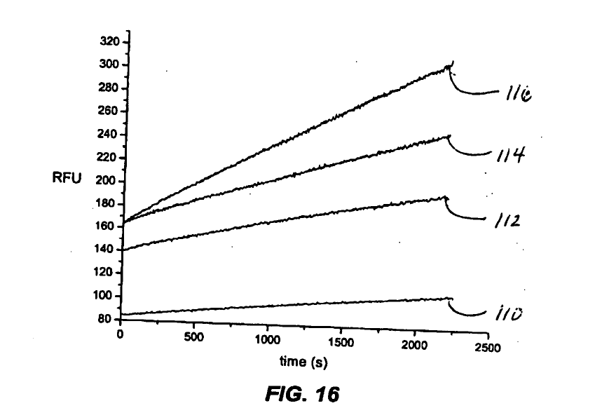

In order to detect the cleavage of compound 60 by PLA2 (FIG. 15) a solution of the SUV's composed of compound 60 (3 ml, 1 μM) was added to a 1 ml quartz cuvette. The initial fluorescence was measured on a Perkin-Elmer LS50 with λex/λem=500/520 nm and ex/em spectral slits set at 2.5/2.5 nm. A solution of PLA2 from bee venom (5 μl, 0.7 unit/ml, 0.5 μg protein/ml, Sigma, Cat. #P9279) was added and the change in fluorescence was detected every second for 2000 s. The temperature was held constant at 25° C. by a circulating water heater. The raw fluorescence data is shown in FIG. 16 in which the initial rate at various initial concentrations of compound 60 is shown at 110, 112, 113, 114, and 116 corresponding to the following concentrations (μM): 50, 100, 200 and 300, respectively.

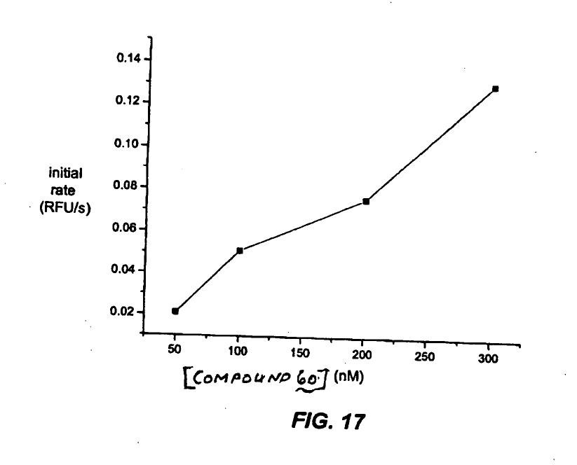

The initial reaction rate (RFU/s) was calculated by linear least squares analysis (Origin 6.0 program) and plotted versus initial compound 60 concentration (FIG. 17). As expected the interfacial enzyme PLA2 does not show Michaelis-Menten behavior (see O. G. Berg et al. in “Interfacial Enzyme Kinetics”, John Wiley and Sons, West Sussex, England (2002)).

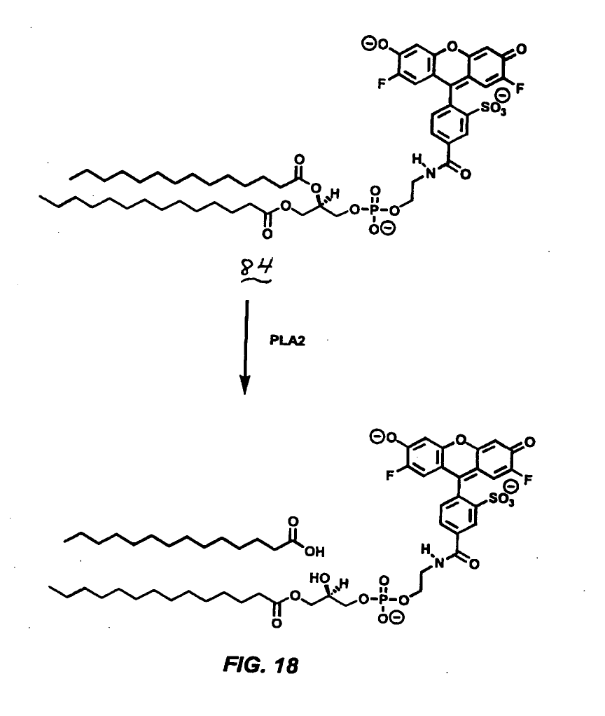

The cleavage of compound 84 by PLA2 (FIG. 18), and HPLC and MS analysis of the products was performed. A solution of compound 84 in methanol (0. 1 ml, 10 mM) was added drop-wise to rapidly stirred aqueous 20 mM TRIS/1 mM CaCl₂ buffer (100 ml, pH 8.0) forming SUV's composed solely of compound 84. A solution of PLA2 (5 μl, 7 units/ml, 5 μg protein/ml) in 20 mM TRIS/1 mM CaCl₂ buffer was added to the stock solution of SUV's composed of compound 84 (1 ml, 10 μM). Conversion of compound 84 to products was detected by reverse phase HPLC (methanol/100 mM TEAA, 80:20 to 90:10, 5 min) (data not shown). After about 30 min, all of compound 84 had been converted to a polar product with the same absorption spectra (diode array detector). Mass spectral analysis of the reaction mixture (not shown) supported the structural assignments.

### 7.4 Example 4

### Analysis of a PLA2 Inhibitor

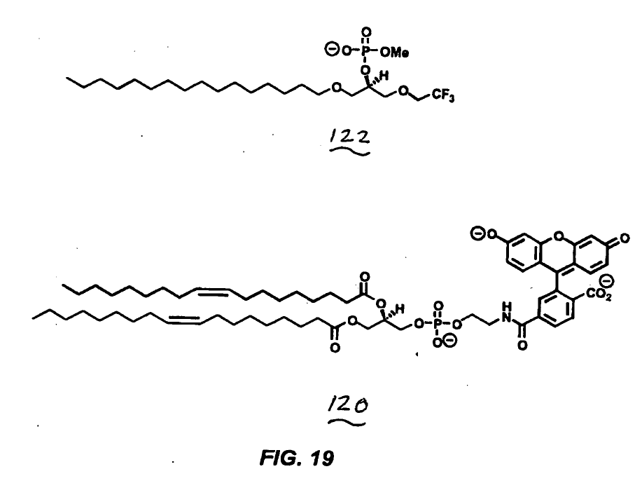

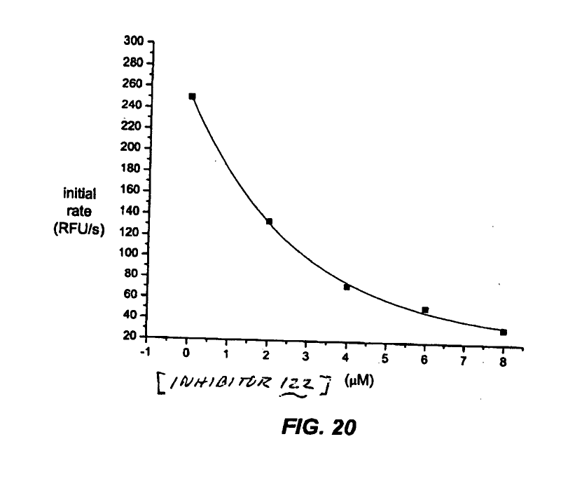

The PLA2 cleavage reaction described in Example 3 was repeated with a concentration of compound 120 (Avanti Polar Lipids, Cat. #790547) fixed at 0.5 μM and with various amounts of inhibitor 122 (Inhibitor MJ33, Sigma, Cat. #M3315) added (FIG. 19). FIG. 20 shows a plot of the initial rate versus concentration of inhibitor 122. The IC₅₀ value was visually approximated to be 2 μM.

### 7.5 Example 5

### Preparation of Monodisperse Liposomes

This example illustrates the preparation of liposomes incorporating a fluorescently-labeled phospholipase substrate. Large unilamellar vesicles (LUV) of diameter 100 nm were prepared by the extrusion method (see S. Chatterjee et al. in Methods in Molecular Biology: Liposome Methods and Protocols, Ed. by S. Basu et al., Humana Press, vol. 199, chapter 1 (2002)). DOPC (12 mg, 15 μmol, Avanti Polar Lipids, Cat. #850375), cholesterol (2 mg, 5 μmol, Avanti Polar Lipids, Cat. #700000) and compound 60 (6 mg, 5 μmol) were dissolved in chloroform (5 ml) in a 50 ml recovery flask. The solvent was thoroughly evaporated under high vacuum to leave a thin film. Aqueous 25 mM Tris/150 mM NaCl buffer (2 ml, pH 7.2, Tris saline) was added and the suspension was subjected to five cycles of freezing (−78° C., dry ice acetone bath) and thawing (40° C.) to hydrate the lipids. The resulting large multilamellar vesicles (LMV) were extruded ten times through 2 stacked 100 nm polycarbonate membranes (Nuclepore track-etch membrane, Whatman, Cat. #110605) using a Lipex™ Extruder (Northern Lipids, Inc., British Columbia, Canada, Cat. #T.001). The LUV were purified by Sephadex™ G-25 M gel filtration (PD-10 column, Amersham Biosciences, Cat. #17-0851-01) eluting with Tris saline. The vesicle size and dispersity was determined by dynamic light scattering using a Nicomp 370 particle size analyzer (Lee Miller, Fine Particle Technology, Menlo Park, Calif.).

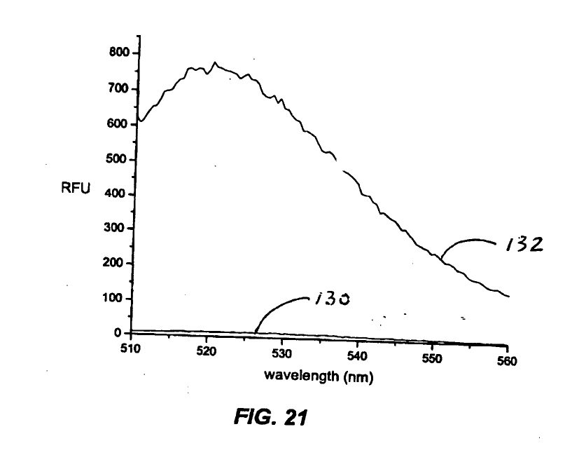

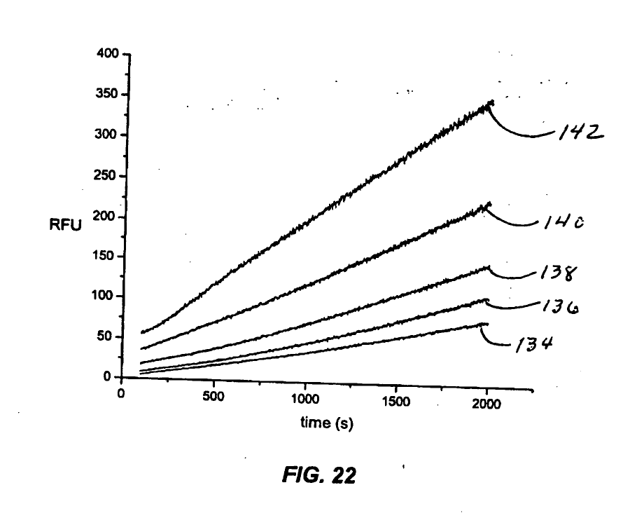

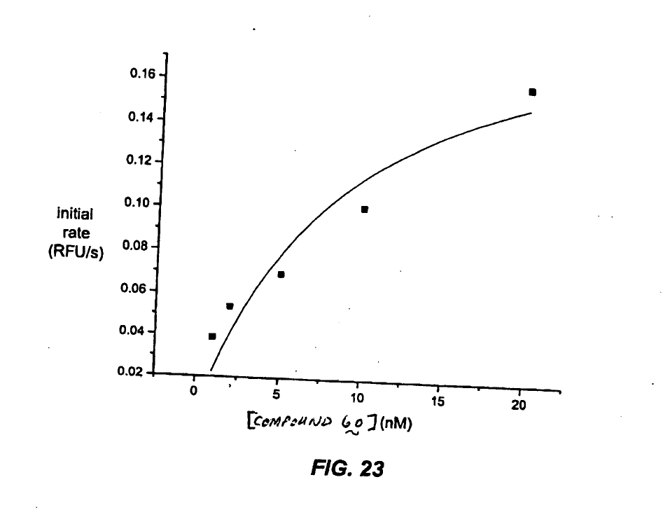

Cleavage of compound 60 in 100 nm monodisperse liposomes by PLC (0.125 nM) was detected using the following method. A solution of PLC from Bacillus cereus (10 μl, 25 units/ml, in Tris saline, Calbiochem Cat. #525186) was added to a 1 ml Tris saline solution of DOPC/cholesterol/compound 60 (60/20/20) LUV (20 nM total lipid, 5 nM concentration of compound 60) in a quartz cuvette. The fluorescence was measured and the data analyzed as in Example 1. FIG. 21 shows the initial 130 and final 132 fluorescence spectra (after a 2000 sec reaction with PLC). There was a 57-fold increase in the RFU. The raw fluorescence data is shown in FIG. 22 in which the initial rate at various initial concentrations of compound 60 is shown at 134, 136, 138, 140, and 142, corresponding to the following concentrations (nM): 1, 2, 5, 10 and 20, respectively. FIG. 23 shows a plot of initial rate as a function of initial concentration of compound 60. The Vmax was 0.21±0.05 RFU/s and the observed Km was 8.6±4.5 nM. The initial rate showed a strong dependence on initial LUV concentration because the liposomes were preformed. In other words the CMC is below the lowest concentration investigated (less than 1 nM). By comparison, the CMC of 100% compound 60 SUV studied in Example 1 was approximately 400 nM.

The effect of the mol % of compound 60 in LUV's on the extent of fluorescence quenching was determined as follows. A solution of PLC (10 μl, 25 units/ml) in Tris saline was added to a solution of 100 nm monodisperse LUVs (1 ml, 100 nM), prepared as described in the paragraph above, in a 1 ml quartz cuvette. The reaction progress was followed until the fluorescence no longer increased (20 to 30 min). The ratio of final to initial fluorescence was calculated to determine the fold increase as a function of mol % of compound 60 in the 100 nm LUV formulation. The results are summarized in Table 5.

TABLE 5

| | DOPC | Cholesterol | Compound 60 | fold |
|---|---|---|---|---|
| Preparation | (mol %) | (mol %) | (mol %) | increase |
| 1 | 40 | 20 | 40 | 100 |
| 2 | 60 | 20 | 20 | 100 |
| 3 | 70 | 20 | 10 | 28 |
| 4 | 75 | 20 | 5 | 5 |

It was observed that the lower the mol % of compound 60 in the LUV the lower the fluorescence quenching.

While the foregoing has presented some embodiments, it is to be understood that these embodiments have been presented by way of example only. It is expected that others will perceive and practice variations-which, though differing from the foregoing do not depart form the spirit and scope of the disclosure as described and claimed herein. All patent applications, patents, and literature references cited in this specification are hereby incorporated by reference in their entirety. In case of conflict or inconsistency, the present description, including definitions, will control.

1. A method of analyzing a sample for the presence of a phospholipase, the method comprising:
contacting a sample suspected of containing a phospholipase with a lipid complex comprising a fluorescently-labeled phospholipase substrate under conditions effective to permit the phospholipase to cleave the fluorescently-labeled phospholipase substrate, wherein the fluorescently-labeled phospholipase substrate comprises a hydrophobic moiety, a phosphate moiety and a fluorescent moiety covalently linked to the phosphate moiety, either directly or via an optional linker; and
detecting the fluorescence of the fluorescent moiety as a function of time, wherein an increase in fluorescence as a function of time correlates with the presence of the phospholipase in the sample.

2. The method of claim 1 in which the fluorescently-labeled phospholipase substrate is present in the lipid complex in an amount effective to quench the fluorescence signal of the fluorescent moiety.

3. The method of claim 1 in which the fluorescently-labeled phospholipase substrate is cleavable by a phospholipase selected from a phospholipase A₁, a phospholipase A₂, a phospholipase B, a phospholipase C and a phospholipase D.

4. The method of claim 1 in which the fluorescent moiety is selected from a xanthene fluorophore, a rhodamine fluorophore, a fluorescein fluorophore, a cyanine fluorophore and a bodipy fluorophore.

5. The method of claim 1 in which the fluorescently-labeled phospholipase substrate has the formula: 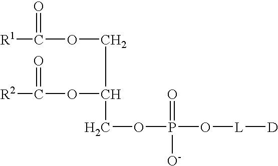
wherein:
R¹ is a saturated or unsaturated alkyl having from 6 to 30 carbon atoms;
R² is a saturated or unsaturated alkyl having from 6 to 30 carbon atoms;
L is a bond or a linker; and
D is a fluorescent fluorophore.

6. The method of claim 5 in which L comprises a linker having the formula —S¹—y—S²—, wherein S¹ and S² are spacing moieties and can be the same length or different lengths, and wherein Y comprises a linkage moiety.

7. The method of claim 5 in which one of R¹ or R² is an alkenyl and the other is an alkanyl.

8. The method of claim 5 in which L is derived from ethanolamine or serine.

9. The method of claim 5 in which D is a fluorescein or a rhodamine fluorophore.

10. The method of claim 5 in which the fluorescently-labeled phospholipase substrate is 1,2-dioleoyl-sn-glycero-3-phosphoethanolamine-N-(Oregon Green 488, 5-isomer) or 1,2-dimyristoyl-sn-glycero-3-phosphoethanolamine-N-(Oregon Green 500, 5-isomer).

11. The method of claim 1 in which the lipid complex is a liposome.

12. The method of claim 1 in which the lipid complex is a micelle.

13. The method of claim 11 in which the liposome further comprises cholesterol.

14. The method of claim 11 in which the liposome encapsulates a fluorescent dye.

15. The method of claim 14 in which the fluorescent dye is capable of quenching the fluorescence of the fluorescent moiety.

16. The method of claim 1 in which the fluorescence signal is detected at one or more discrete time points.

17. The method of claim 1 in which the fluorescence signal is detected substantially continuously.

18. A method of detecting a cleavage activity of a phospholipase, the method comprising:
contacting a phospholipase with a lipid complex comprising a fluorescently-labeled phospholipase substrate under conditions effective to permit the phospholipase to cleave the phospholipase substrate, wherein the phospholipase substrate comprises a hydrophobic moiety, a phosphate moiety and a fluorescent moiety covalently linked to the phosphate moiety either directly or via an optional linker; and
detecting the fluorescence of the fluorescent moiety as a function of time, wherein an increase in fluorescence correlates with the presence of a cleavage activity of the phospholipase.

19. The method of claim 18 in which the fluorescently-labeled phospholipase substrate is present in the lipid complex in an amount effective to quench the fluorescence signal of the fluorescent moiety.

20. The method of claim 18 in which the fluorescently-labeled phospholipase substrate is cleavable by a phospholipase selected from a phospholipase A₁, a phospholipase A₂, a phospholipase C and a phospholipase D.

21. The method of claim 18 in which the fluorescent moiety is selected from a xanthene fluorophore, a rhodamine fluorophore, a fluorescein fluorophore, a cyanine fluorophore and a bodipy fluorophore.

22. The method of claim 18 in which the fluorescently-labeled phospholipase substrate has the formula: 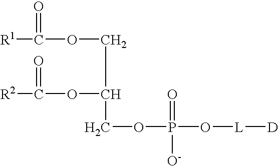
wherein:
R¹ is a saturated or unsaturated alkyl having from 6 to 30 carbon atoms;
R² is a saturated or unsaturated alkyl having from 6 to 30 carbon atoms;
L is a bond or a linker; and
D is a fluorescent fluorophore.

23. The method of claim 22 in which L comprises a linker having the formula —S¹—Y—S²—, wherein S¹ and S² are spacing moieties and can be the same length or different lengths, and wherein Y comprises a linkage moiety.

24. The method of claim 22 in which one of R¹ or R² is an alkenyl and the other is an alkanyl.

25. The method of claim 22 in which L is derived from ethanolamine or serine.

26. The method of claim 22 in which D is a fluorescein or a rhodamine fluorophore.

27. The method of claim 22 in which the fluorescently-labeled phospholipase substrate is 1,2-dioleoyl-sn-glycero-3-phosphoethanolamine-N-(Oregon Green 488, 5-isomer) or 1,2-dimyristoyl-sn-glycero-3-phosphoethanolamine-N-(Oregon Green 500, 5-isomer).

28. The method of claim 18 in which the lipid complex is a liposome.

29. The method of claim 18 in which the lipid complex is a micelle.

30. The method of claim 28 in which the liposome further comprises cholesterol.

31. The method of claim 28 in which the liposome encapsulates a fluorescent dye.

32. The method of claim 31 in which the fluorescent dye is capable of quenching the fluorescence of the fluorescent moiety.

33. The method of claim 18 in which the fluorescence signal is detected at discrete time points.

34. The method of claim 18 in which the fluorescence signal is detected substantially continuously.

35. A method of identifying a modulator of a phospholipase, comprising the steps of:
contacting a phospholipase with a lipid complex comprising a fluorescently-labeled phospholipase substrate in the presence of a candidate compound under conditions effective to permit the phospholipase to cleave the phospholipase substrate, wherein the phospholipase substrate comprises a hydrophobic moiety, a phosphate moiety and a fluorescent moiety covalently linked to the phosphate moiety either directly or via an optional linker; and
detecting the fluorescence of the fluorescent moiety as a function of time, wherein an increase or decrease in the quantity or rate of accumulation of fluorescence as compared to a control identifies the compound as a modulator of the phospholipase

36. The method of claim 35 in which the fluorescently-labeled phospholipase substrate is present in the lipid complex in an amount effective to quench the fluorescence signal of the fluorescent moiety.

37. The method of claim 35 in which the fluorescently-labeled phospholipase substrate is cleavable by a phospholipase selected from a phospholipase A₁, a phospholipase A₂, a phospholipase C and a phospholipase D.

38. The method of claim 35 in which the fluorescent moiety is selected from a xanthene fluorophore, a rhodamine fluorophore, a fluorescein fluorophore, a cyanine fluorophore and a bodipy fluorophore.

39. The method of claim 35 in which the fluorescently-labeled phospholipase substrate has the formula: 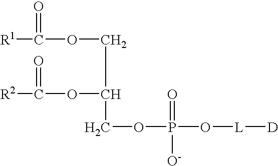
wherein:
R¹ is a saturated or unsaturated alkyl having from 6 to 30 carbon atoms;
R² is a saturated or unsaturated alkyl having from 6 to 30 carbon atoms;
L is a bond or a linker; and
D is a fluorescent fluorophore.

40. The method of claim 39 in which L comprises a linker having the formula —S¹—Y—S²—, wherein S¹ and S² are spacing moieties and can be the same length or different lengths, and wherein Y comprises a linkage moiety.

41. The method of claim 39 in which one of R¹ or R² is an alkenyl and the other is an alkanyl.

42. The method of claim 39 in which L is derived from ethanolamine or serine.

43. The method of claim 39 in which D is a fluorescein or a rhodamine fluorophore.

44. The method of claim 39 in which the fluorescently-labeled phospholipase substrate is 1,2-dioleoyl-sn-glycero-3-phosphoethanolamine-N-(Oregon Green 488, 5-isomer) or 1,2-dimyristoyl-sn-glycero-3-phosphoethanolamine-N-(Oregon Green 500, 5-isomer).

45. The method of claim 35 in which the lipid complex is a liposome.

46. The method of claim 35 in which the lipid complex is a micelle.

47. The method of claim 45 in which the liposome further comprises cholesterol.

48. The method of claim 45 in which the liposome encapsulates a fluorescent dye.

49. The method of claim 48 in which the fluorescent dye is capable of quenching the fluorescence of the fluorescent moiety.

50. The method of claim 35 in which the fluorescence signal is detected at discrete time points.

51. The method of claim 35 in which the fluorescence signal is detected substantially continuously.

52. The method of claim 35 which is carried out to identify an activator of the phospholipase.

53. The method of claim 35 which is carried out to identify an antagonist of the phospholipase.

54. A method of identifying a modulator of a phospholipase, comprising the steps of:
contacting a phospholipase, a lipid complex comprising a fluorescently-labeled phospholipase substrate and a candidate compound under conditions effective to permit the phospholipase to cleave the phospholipase substrate, wherein the phospholipase substrate comprises a hydrophobic moiety, a phosphate moiety and a fluorescent moiety covalently linked to the phosphate moiety either directly or via an optional linker; and
detecting the fluorescence of the fluorescent moiety as a function of time, wherein an increase or decrease in the quantity or rate of accumulation of fluorescence as compared to a control reaction identifies the compound as a modulator of the phospholipase.

55. A composition comprising a phospholipase inhibitor and a lipid complex, said lipid complex comprising a phospholipid and a fluorescently-labeled phospholipase substrate, wherein the phospholipase substrate comprises a hydrophobic moiety, a phosphate moiety and a fluorescent moiety covalently attached to the phosphate moiety either directly or by way of an optional linker, and is included in the lipid complex in an amount effective to quench the fluorescence signal of the fluorescent moiety.

56. The lipid complex of claim 55 in which the fluorescent moiety is selected from a xanthene fluorophore, a fluorescein fluorophore, a rhodamine fluorophore and a cyanine fluorophore.

57. The lipid complex of claim 55 in which the fluorescent moiety is an energy transfer fluorophore.

58. The lipid complex of claim 55 which is in the form of a liposome.

59. The lipid complex of claim 58 which further includes cholesterol.

60. The lipid complex of claim 59 in which the cholesterol is in the range of 5 mol % to 80 mol %.

61. The lipid complex of claim 59 in which the amount of the cholesterol ranges from 10 to 50 mol %.

62. The lipid complex of claim 55 which encapsulates a fluorescent dye capable of quenching the fluorescent signal of the fluorescent moiety.

63. The lipid complex of claim 55 in which the phospholipid is selected from the group consisting of phosphatidylcholine, phosphatidylethanolamine, phosphatidic acid, phosphatidylserine, phosphatidylinositol, phosphatidylglycerol, sphingomyelin, cardiolipin, and mixtures thereof.

64. The lipid complex of claim 55 in which the amount of the phospholipase substrate ranges from 1 to 100 mol %.

65. The lipid complex of claim 55 comprising cholesterol and dioleoyl-phosphtidylcholine.

66. The lipid complex of claim 55 in which the phospholipid substrate comprises the formula: 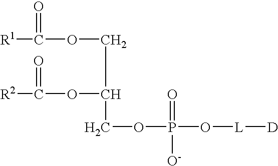
or a salt thereof, wherein:
R¹ is a saturated or unsaturated hydrocarbon comprising from 6 to 30 carbon atoms;
R¹ is a saturated or unsaturated hydrocarbon comprising from 6 to 30 carbon atoms;
L is a bond or a linker; and
D is a fluorescent moiety.

67. The complex of claim 66 in which L comprises a linker having the formula —S¹—Y—S²—, wherein S¹ and S² are spacing moieties and can be the same length or different lengths, and wherein Y comprises a linkage moiety.

68. The lipid complex of claim 66 in which D is a fluorescein or a rhodamine fluorophore.

69. The lipid complex of claim 66 in which one of R¹ or R² is a saturated alkyl and the other is an unsaturated alkyl.

70. The lipid complex of claim 66 in which one of R¹ or R² is an alkanyl and the other is an alkenyl.

71. The lipid complex of claim 66 in which the fluorescently-labeled phospholipase substrate is 1,2-dioleoyl-sn-glycero-3-phosphoethanolamine-N-(Oregon Green 488, 5-isomer) or 1,2-dimyristoyl-sn-glycero-3-phosphoethanolamine-N-(Oregon Green 500, 5-isomer).

72. A kit for use in a phospholipase reaction, said kit comprising: at least one fluorescently labeled phospholipase cleavage substrate for detecting a target phospholipase and a buffer for preparing a reaction mixture that facilitates the phospholipase reaction.

73. The kit of claim 72 further including a phospholipase.

74. The kit of claim 73 wherein the phospholipase substrate is included in a lipid complex.

75. The kit of claim 74 wherein the lipid complex includes a fluorescent dye.

76. The kit of claim 75 wherein the lipid complex includes a fluorescence quenching compound.

77. The kit of claim 72 further including a modulator of a phospholipase activity.

78. The kit of claim 77 wherein said modulator is a phospholipase antagonist.

79. The kit of claim 78 wherein said antagonist is tricyclodecan-9-yl-xanthogenate or MJ33.

80. The kit of claim 77 wherein said modulator is a phospholipase activator.

81. The kit of claim 76 wherein said phospholipase is an enzyme standard.

82. The kit of claim 76 wherein said phospholipase is selected from the group consisting of a phospholipase A₁, a phospholipase A₂, a phospholipase C, a phospholipase D, and mixtures thereof.

83. The kit of claim 76 further including instructions for carrying out the method of claim 1.

84. The method of any of claims 1, 18 or 35 in which the fluorescently-labeled substrate is capable of fluorescing in aqueous medium.

85. The composition of claim 55 in which the fluorescently-labeled substrate is capable of fluorescing in aqueous medium.

86. The kit of claim 72 in which the fluorescently-labeled substrate is capable of fluorescing in aqueous medium.
